SEQUENCE LISTING (12 sequences)

SEQ ID NO: 1 - DNA, 20 bp, Artificial Sequence
  primer HPV16 E6-FW
gcaacagttactgcgacgtg

SEQ ID NO: 2 - DNA, 20 bp, Artificial Sequence
  primer HPV16 E6-RV
ggacacagtggcttttgaca

SEQ ID NO: 3 - DNA, 20 bp, Artificial Sequence
  primer HPV16 E7-FW
gatggtccagctggacaagc

SEQ ID NO: 4 - DNA, 20 bp, Artificial Sequence
  primer HPV16 E7-RV
gtgcccattaacaggtcttc

SEQ ID NO: 5 - DNA, 22 bp, Artificial Sequence
  primer K19-FW
gaggtggattccgctccgggca

SEQ ID NO: 6 - DNA, 21 bp, Artificial Sequence
  primer K19-RV
atcttcctgtccctcgagcag

SEQ ID NO: 7 - DNA, 20 bp, Artificial Sequence
  primer MUC1-FW
gtaccatcaatgtccacgac

SEQ ID NO: 8 - DNA, 20 bp, Artificial Sequence
  primer MUC1-RV
ctacgatcggtactgctagg

SEQ ID NO: 9 - DNA, 20 bp, Artificial Sequence
  primer alpha-SM Actin-FW
ggaatcctgtgaagcagctc

SEQ ID NO: 10 - DNA, 24 bp, Artificial Sequence
  primer alpha-SM Actin-RV
cacagttgtgtgctagagacagag

SEQ ID NO: 11 - DNA, 18 bp, Artificial Sequence
  primer GAPDH-FW
gaaggtgaaggtcggagt

SEQ ID NO: 12 - DNA, 20 bp, Artificial Sequence
  primer GAPDH-RV
gaagatggtgatgggatttc

(19) United States
(12) Patent Application Publication
(10) Pub. No.: US 2005/0244388 A1
(43) Pub. Date: Nov. 3, 2005

(54) Suprabasal breast cell with stem cell properties

(75) Inventors: Lone Ronnov-Jessen Petersen, Vaerlose (DK); Ole William Petersen, Vaerlose (DK); Thorarinn Godjonsson, Reykjavik (IS); Rene Villadsen, Frederikssund (DK); Mona J. Bissell, Berkeley, CA (US)

Correspondence Address: MERCHANT & GOULD PC, P.O. BOX 2903, MINNEAPOLIS, MN, 55402-0903, US

(73) Assignee: KOBENHAVNS UNIVERSITET, KOBENHAVN N (DK)

(21) Appl. No.: 10/501,289

(22) PCT Filed: Jan. 16, 2003
(86) PCT No.: PCT/EP03/00518

§ 371 Date: Jun. 29, 2005

(30) Foreign Application Priority Data
Jan. 17, 2002 (DK) PA 200200079

(51) Int. Cl. C12Q 1/00; A61K 48/00; C12N 5/08

(52) U.S. Cl. 424/93.21; 435/4; 435/366

(57) ABSTRACT

A method for isolating cells which have a suprabasal position and expresses epithelial specific antigen but no sialomucin is provided. The isolated cells are shown to share many of the properties expected of a mammary gland stem cell. Three permanent cell lines that are capable of proliferating and capable of differentiating into cells of mammary gland luminal epithelial and myoepithelial cell lineages were established. Such cells form elaborate branching structures resembling uncultured terminal duct lobular units both by morphology and marker expression both in vitro and in vivo. Evidence is provided that these keratin K19 expressing cells most probably is the cells in which breast cancer arises. Thus they constitute a model not only for the developing breast, but also for the development of breast cancer. Also disclosed are the uses of such isolated cells or the cell lines as a model system of the mammary gland for pharmacological studies and their uses in tissue repair or transplantation.

### FIELD OF INVENTION

The present invention relates to the isolation of a new at least bi-potent cell type from luminal epithelial cells of a mammary gland and its establishment as an immortalised cell line which is capable of proliferating and capable of differentiating into cells of mammary gland luminal epithelial and myoepithelial cell lineages. The invention furthermore relates to the uses of the isolated cells or the cell lines as a model system of the mammary gland and to the uses in tissue repair or transplantation.

### GENERAL BACKGROUND

Understanding how the normal human breast develops and which cell compartment becomes neoplastic by necessity is dependent on the isolation of relevant cells as the true targets of human breast carcinogenesis and progression. More than two decades ago it was proposed that human breast cancer originates from the luminal epithelial lineage within the terminal duct lobular units (TDLU), a basic mammary structure consisting of a branching ductal-alveolar system lined by an inner layer of luminal epithelial cells and an outer layer of myoepithelial cells.

Recently, others and the present inventors have provided some evidence that the stem cells of the human and mouse breast gland may be contained within the luminal epithelial lineage (Smith 1996; Stingl et al. 1998; Péchoux et al. 1999; Smalley et al. 1999; Stingl et al. 2001). However, detailed ultrastructural characterisations of the rodent and human breast gland in situ have led to the widely discussed hypothesis that the stem cell is a “basal cell” with clear cytoplasm (for review see (Smith and Chepko 2001). In particular, the important work of Smith and colleagues in the mouse mammary gland has outlined an elaborate morphological tree identifying the actual stem cell (small light cells; SLC), a first degree progenitor cell (not distinct from SLC), a second degree progenitor which is still multipotent (undifferentiated large light cell, ULLC), and then two compartments of non-dividing, pre-luminal- and pre-myoepithelial cells which gradually mature into the fully differentiated lineages. At the ultrastructural level, the SLC never reaches the acinus lumen and only a fraction of the ULLC does so (Smith and Chepko 2001). Since sialomucin is the most prominent marker of luminal epithelial cells and is exclusively expressed on the apical surface of luminal epithelial cells, this would imply that some epithelial cells (not facing the lumen) are sialomucin negative. Nevertheless, there is reason to believe that these cells are indeed full members of the luminal epithelial lineage. The cells on the basal side of multilayered breast ducts express several luminal epithelial markers including simple epithelial keratins and epithelial specific antigen (ESA), but no markers of the myoepithelial lineage such as α-smooth muscle actin or Common Acute Lymphoblastic Leukaemia Antigen (CALLA). Cells with this phenotypic profile have in fact been observed in cultures of human reduction mammoplasties and shown to be bi-potent—suggesting a stem cell potential (Stingl et al. 1998). However, further characterisation of the putative stem cells to show the full potential of generating TDLU have not been pursued due to a limited growth potential in primary culture.

### SUMMARY OF THE INVENTION

The problem to be solved by the present invention was to isolate the cells of the mammalian breast gland which are able to form terminal duct lobular units (TDLUs) in order to provide a cell culture which may serve as a model for breast gland development. Since breast cancer is suspected to originate in the cells forming the TDLUs such cells could, if isolated, also serve as a model for breast cancer development.

The solution was to define and then to isolate such a cell type. Based on the literature and our own extensive observations the present inventors reasoned that a breast epithelial cell type which expresses epithelial specific antigen (ESA) but little or no sialomucin seemed a good candidate to the precursor cell forming TDLU.

The present inventors show that a minor population of suprabasal-positioned, subluminal, ESA-positive and sialomucin-negative/weakly positive cells (ESA⁺/MUC⁻ cells) indeed exists in vivo.

To accomplish the isolation of such a cell, the present inventors isolated distinct cell populations using immunomagnetic sorting by first removing the sialomucin expressing cells and then isolating sialomucin-negative cells that expresses ESA. The present Inventors show that the disclosed method of isolation consistently results in cells that possess properties expected of TDLU precursor cells. Accordingly, the invention provides in a first aspect a method for isolation of an at least bi-potent mammary gland tissue cell, comprising the steps of:

(i) separating said tissue into two or more different cell types

(ii) culturing each of said different cell types under cell differentiation conditions and

(iii) selecting the cell type(s) that is/are capable of differentiating into at least two morphologically and/or phenotypically different cell types.

The isolated suprabasal-derived ESA⁺/MUC⁻ cells were of luminal epithelial lineage because they expressed tight junction proteins and exhibited a high transepithelial electrical resistance on transwell filters. However, in contrast to luminal epithelial cells with strong sialomucin expression they had a striking ability to form the entire TDLUs inside a three-dimensional reconstituted basement membrane and in nude mice and could generate myoepithelial cells, as well as luminal epithelial cells. Thus these cells share many of the properties expected of a mammary stem cell. Accordingly, an important aspect of the invention is to provide an isolated cell, derived from luminal epithelial cells of a mammary gland, which is capable of proliferating and capable of differentiating into cells of mammary gland luminal epithelial and myoepithelial cell lineages. In a further aspect, the invention pertains to a cell population composed of such cells.

In further aspects of the invention, immortalised cell lines derived from said isolated cells that are capable of differentiating into cells of mammary gland luminal epithelial and myoepithelial cell lineages are provided.

To obtain such permanent cell lines with stem cell properties the cells were immortalised with HPV (human papilloma virus)-16 E6/E7. Three cell lines that are capable of proliferating and capable of differentiating into cells of mammary gland luminal epithelial and myoepithelial cell lineages were established (D492, D490 and TH69). One of these (D492) was deposited in accordance with the provisions of the Budapest Treaty on the International Recognition of the Deposit of Micro-organisms for the Purposes of Patent Procedure at Deutsche Sammlung von Mikroorganismen und Zellkulturen GmbH (DSMZ) and has obtained the accession number DSM ACC 2529. Also non-immortalised cell suprabasal-derived epithelial cell cultures obtained by the disclosed method of isolation (TH82 and TH95) possess stem cell properties.

Importantly, suprabasal-derived ESA⁺/MUC⁻ cells differ from luminal-derived ESA⁺/MUC⁺ cells by the expression of keratin K19. The present inventors have localised a subpopulation of luminal epithelial cells in the normal breast in situ by the restricted expression of keratin K19 similarly to the cells of the invention. The present inventors propose that these cells are indeed candidate stem cells or multipotent progenitor cells of the mammalian breast gland. Thus an important embodiment of the invention is an immortalised cell line that is derived from said isolated cells that are capable of differentiating into cells of mammary gland luminal epithelial and myoepithelial cell lineages and which comprises cells that are positive staining for keratin K19.

To the best of our knowledge the invention is the first in vitro model of the developing human breast gland model.

The present inventors propose that, since more than 90% of human breast carcinomas are keratin K19 positive and originate from TDLU, the present inventors have identified the cellular origin of most human breast cancers. Thus a further aspect of the invention is to use the at least bi-potent breast cells of the invention, and which shares many of the characteristics of the putative cellular origin of most human breast cancers, as an in vitro model for the study of breast cancer development and in particular to provide a method for testing the carcinogenic effect, if any, of a substance on mammary gland epithelial cells, the method comprising:

(i) culturing said cells in a growth medium which maintains the cells as non-transformed cells;

(ii) adding the agent, compound or factor under test to the cell culture; and

(iii) determining the neoplastic response, if any, of the so contacted cells by changes in morphology, tumorigenicity in animals, mRNA expression and/or antigen expression as well as other changes which is associated with carcinogenicy.

In further aspects of the invention the isolated suprabasal-derived ESA⁺/MUC⁻ cells are used as an in vitro model of the developing human breast gland to screen for pharmaceutical interesting or toxic substances. Thus the invention provide a method for testing the toxic effect, if any, of a substance on mammary gland epithelial cells, comprises:

(i) culturing or maintaining said cells in a non-toxic medium;

(ii) adding the substance to be tested to the medium; and

(iii) determining the response, if any, of the cells, including changes in cell growth rate, cell death rate, apoptosis, cell metabolism, inter- as well as intra-cellular communication, morphology, mRNA or protein expression and antigen expression.

In further aspects the cells of the invention are used to provide mutatis mutandis a similar three step method for testing the ability, if any, of a substance to modulate the differentiation of non-terminal differentiated mammary gland epithelial cells and a three step method for screening a substance for its ability, if any, to interact with a cellular protein.

In a further aspect of the invention the cells isolated by the method disclosed in the present application are expanded and provides a method of transplanting a vertebrate host with said cells, comprising the step of introducing the cell into the vertebrate host. In yet a further development of this aspect said cells provide a method of in vivo administration of a protein or gene of interest to an individual in need thereof, comprising the step of transfecting the cell-population with a vector comprising DNA or RNA which expresses the protein or gene of interest and introducing the transfected cell into said individual. In yet a further development of the invention said cells provide a method of tissue repair or transplantation in mammals, comprising administering to a mammal a therapeutically effective amount of cells or tissues derived therefrom.

### DETAILED DISCLOSURE OF THE INVENTION

The object of the present invention is to provide a cell culture, which is able to form a structure similar to the terminal duct lobular units (TDLUs) of the mammary gland in order to provide a cell culture, which serves as a model for the normal breast gland development. In the present context the “terminal duct lobular units (TDLUs)” of the mammary gland is defined as the basic mammary structure consisting of a branching ductal-alveolar system lined by an inner layer of luminal epithelial cells and an outer layer of myoepithelial cells.

Based on experiments with cells in culture it has been suggested that a putative “stem” cell of the human TDLUs may be contained within the luminal epithelial lineage of cells and characterised by a positive staining for the luminal epithelial marker (ESA⁺) and a negative or weakly positive staining for sialomucin (MUC⁻). In Example 1 data is presented showing that such an MUC⁻/ESA⁺ luminal epithelial cell type exists in the human breast in vivo and that they are suprabasally positioned.

Example 2 describes the isolation, immortalisation and characterisation of suprabasal-derived epithelial “stem” or “progenitor” cells as well as the isolation of luminal cells without “stem-cell” properties. In the present context “stem cell” or “progenitor cell” is defined as an at least bi-potent mammary gland tissue cell that is able to differentiate into cells of mammary gland luminal epithelial and myoepithelial cell lineages. The progenitor cells were isolated the from suprabasal-positioned luminal epithelial cells of the mammary gland by immunomagnetic cell sorting exploiting the assumed MUC⁻/ESA⁺ phenotype of the wanted cell type.

In the present context the term “suprabasal” is defined as abluminal and separated from the basement membrane by a layer of myoepithelial cells. The term “suprabasal-positioned” is used to describe cells that are positioned between the luminal and the myoepithelial cell layer, and the term “suprabasal-derived” is used to describe cells that are isolated from the suprabasal layer of luminal cells of the TDLUs.

In a particular useful embodiment of the invention an isolated cell, derived from luminal epithelial cells of a mammary gland, which is capable of proliferating and capable of differentiating into cells of mammary gland luminal epithelial and myoepithelial cell lineages and which is isolated from suprabasal luminal epithelial cells of the mammary gland is provided.

In one embodiment of the invention, which is illustrated in example 2, the isolated cell is a human cell. It is however contemplated that a similar stem or progenitor cell also can be isolated by the procedure described in example 2 from mamma of other species resulting in a cell selected from the group consisting of a rodent cell, a porcine cell, a ruminant cell, a bovine cell, a caprine cell, a equine cell, a canine cell, a ovine cell, a feline cell and a primate cell.

As illustrated in example 2 such an isolated cell is capable of forming a cell culture comprising cells which are positive staining for the luminal epithelial marker ESA (ESA+) and negative or weakly positive staining for sialomucin (MUC−), so-called ESA⁺/MUC− cells. By “positive staining” the present inventors refer to clearly visible cells after staining with the primary antibody in a 1:200 dilution for immunoperoxidase or a 1:10 dilution for immunofluorescence.

In a preferred embodiment of the invention said isolated cell is immortalised. An immortalised cell is a prerequisite for establishing a permanent cell line. Whereas rodent cells are relative prone to undergo even apparently spontaneous immortalisation human cells are remarkably resistant to immortalisation. Thus a particular useful embodiment of the present invention is an immortalised ESA+/MUC− cell. However cells, including human cells, may be immortalised by a number of procedures. The literature provides examples of cells being immortalised as a result of exposure to various chemicals including carcinogens and tumour promoters (Balmain and Harris, 2000) and as a result of the introduction of a nucleic acid molecule encoding an immortalising polypeptide (Katakura et al., 1998). By “a nucleic acid molecule encoding an immortalising polypeptide” the present inventors refer to a nucleic acid molecule that codes for a polypeptide the expression of which either alone or in combination with other polypeptides result in the immortalisation of the respective cell. In the present context an immortalised cell is defined as a cell capable of in vitro growth for preferably at least 50 doublings, more preferably at least 75 doublings, and most preferably at least 100 doublings. This is to be compared with the normal situation where senescence occurs after 30 doublings. Furthermore a distinct telomerase activity which is absent from finite life span breast epithelial cells can be used to define immortalised cells (Stampfer et al, 2001). Thus one specific embodiment of the invention is an immortalised cell line, wherein the immortalising step comprises transfecting the cells with a nucleic acid molecule encoding an immortalising polypeptide.

According to presently preferred embodiment, the immortalisation is performed by introducing into the cells a nucleic acid vector comprising at least one nucleic acid sequence encoding an oncogenic polypeptide selected from the group of transforming oncogenes which has been shown to be able to immortalise cells either alone or in combination with other genes. Examples of such genes are c-myc, N-myc, L-myc, SV40 large T antigen, adenovirus E1A, papillomaviruses E6 and E7, polyoma Large T gene, erbA, myb, fos, jun, p53 or an oncogenic part of any one thereof. However, also nucleotide sequences from Epstein-Barr virus, Herpes virus and certain other virus has been implicated in the immortalisation of cells.

Recently, a number of reports have demonstrated the immortalisation potential of human papillomavirus-16 E6 and E7 genes (HPV16-E6/E7). Thus according to an embodiment of the invention, immortalised cell line is characterised by an immortalising step that comprises transfecting the cells with a nucleic acid molecule encoding a papillomavirus polypeptide selected from the group consisting of E6, E7 and a nucleic acid molecule comprising E6 and E7.

The major concern with this technique is that while it has been shown that immortality is achieved by the inactivation of p53 and retinoblastoma protein (RB), these may not be the only affected molecules and other cellular functions may also be affected (for review see (Zwerschke and Jansen-Durr 2000). Evidence suggests, however, that human cells derived from E6/E7 immortalisation retain much of their original phenotype. In organotypic cultures of endocervical cells, which is a target organ in vivo for HPV-16 infection, the cells appeared normal with ordinary stratification and production of a cornified layer. Also, normal adult human pancreatic epithelial cells transfected with E6/E7 remained polarized on collagen gels did not grow in soft agar and expressed typical simple keratins. Thus, whereas E6 and E7 readily induce a neoplastic transformation of rodent cells when transferred into the cells, human cells appear to be significantly more robust. Human cells transfected with E6 and E7 do not form tumours in nude mice even after more than 100 passages in culture (Willey et al. 1991; Band 1995;). Immortalisation of normal human breast cells with either E6 or E7 did not lead to aberrant functional behaviour in luminal or myoepithelial cells tested (Wazer et al. 1995). It has been reported that breast cells lose keratin K19 expression as a consequence of E7-immortalisation (Spancake et al. 1999), but this was not confirmed by the present inventors (see below) or others (Wazer et al. 1995). Finally, human salivary gland cells transduced with E6/E7 remained diploid or near-diploid without a general destabilisation of the karyotype. The present inventors have confirmed these studies, and have found that the transduced cells are non-tumorigenic and have a diploid karyotype (46, XX) even after more than one year in culture and 25 passages. Thus it appears that the method used for immortalising the isolated MUC⁻/ESA⁺ progenitor cells and which comprises transforming the cells with a retroviral vector including an expression cassette comprising a nucleic acid molecule encoding a papillomavirus polypeptide E6 and E7, and selecting the immortalised cells results in a non-tumorigenic cell line.

As used herein, the expressions “transforming” and “transducing” are used interchangeably and refer to the introduction of DNA into a recipient cell, irrespective of the method used for the introduction. Also the term “transfection” refers to the introduction of DNA into a recipient cell. But whereas the term “transduction” typically refers to a method of introduction which comprises virus particles, the term “transfection” may refer both to methods which involves virus as well as methods which rely on virus-free compositions containing specific nucleic acids. In relation to eucaryotic cells the term “transfection” normally refers to the introduction of virus-free DNA compositions into a recipient cell. In the present context the expressions “transforming”, “transduction”, and “transfection” are used interchangeably to describe the introduction of DNA into a recipient cell. Note however, that the terms “preneoplastic transformation” or “malignant transformation” in general and also in the present context refer to the process wherein a cell changes on or more phenotypically traits that characterise a neoplastic cell. One such trait is the ability to form tumours in syngeneic or immuno-incompetent animals.

A “vector” is a composition which can transduce, transform or infect a cell, thereby causing the cell to express vector encoded nucleic acids and, optionally, proteins other than those native to the cell, or in a manner not native to the cell. A vector includes a nucleic acid (ordinarily RNA or DNA) to be expressed by the cell. A vector optionally includes materials to aid in achieving entry of the nucleic acid into the cell, such as a retroviral particle.

In a preferred embodiment of the invention the cells are immortalised by transforming the cells with a retroviral vector. By the term “retroviral vector” are meant vectors that comprises retroviruses. Most retroviral vectors are based on murine retrovirus. They can carry 6 to 7 kb of foreign DNA (promoter+cDNA). Thus, according to the said preferred embodiment, the purified precursor cells were immortalised by a procedure which comprises transforming the cells with at least one retroviral vector including an expression cassette comprising a nucleic acid molecule encoding a papillomavirus polypeptide selected from the group consisting of E6, E7 and a nucleic acid molecule comprising E6 and E7, and selecting the immortalised cells.

The invention further relates to a method of immortalisation that is based on other vectors for instance vectors based on adenovirus, adeno-associated virus, papilloma virus and plasmids.

In all cases the vector must comprise an expression cassette. By the term “expression cassette” is meant a nucleic acid sequence that comprises the elements necessary to express an inserted cDNA in the host of interest. For a mammalian cell host, such a vector typically contains a powerful promoter coupled to an enhancer, a cloning site, and a polyadenylation signal. In addition to the expression cassette, several expression vectors also contain a selectable marker gene such as DHFR or NeoR, which aids in the generation of stable cell lines. The expression cassette may contain one or more unrelated DNA sequences encoding one or more peptides of interest.

In a particular, the immortalising step can be performed by transforming the cells with retrovirus-containing supernatant from the PA317 LXSN HPV16E6E7 packaging cell line (CRL-2203, ATCC, Rockville, Md.) cell line and selecting the immortalised cells. Thus a preferred embodiment of present invention is an immortalised cell line, wherein the immortalising step is performed by transforming the cells with retrovirus-containing supernatant from the PA317 LXSN HPV16E6E7 cell line and selecting the immortalised cells.

As described in example 3 only the suprabasal-derived cell lines were able to develop into mixed cultures of luminal epithelial and myoepithelial cells.

The fact that the suprabasal-derived epithelial cell lines continued for more than 50 passages to generate subpopulations of ESA⁺/MUC⁺ and ESA⁻/MUC⁻ cells as well as ESA⁺/MUC⁻ cells make them strong candidates for stem cells or multipotent progenitors of the breast, as the ESA⁺/MUC⁺ cells represent differentiated luminal epithelial cells, while the ESA⁻/MUC⁻ cells represent myoepithelial cells, which are ESA⁻/MUC⁻ in vivo.

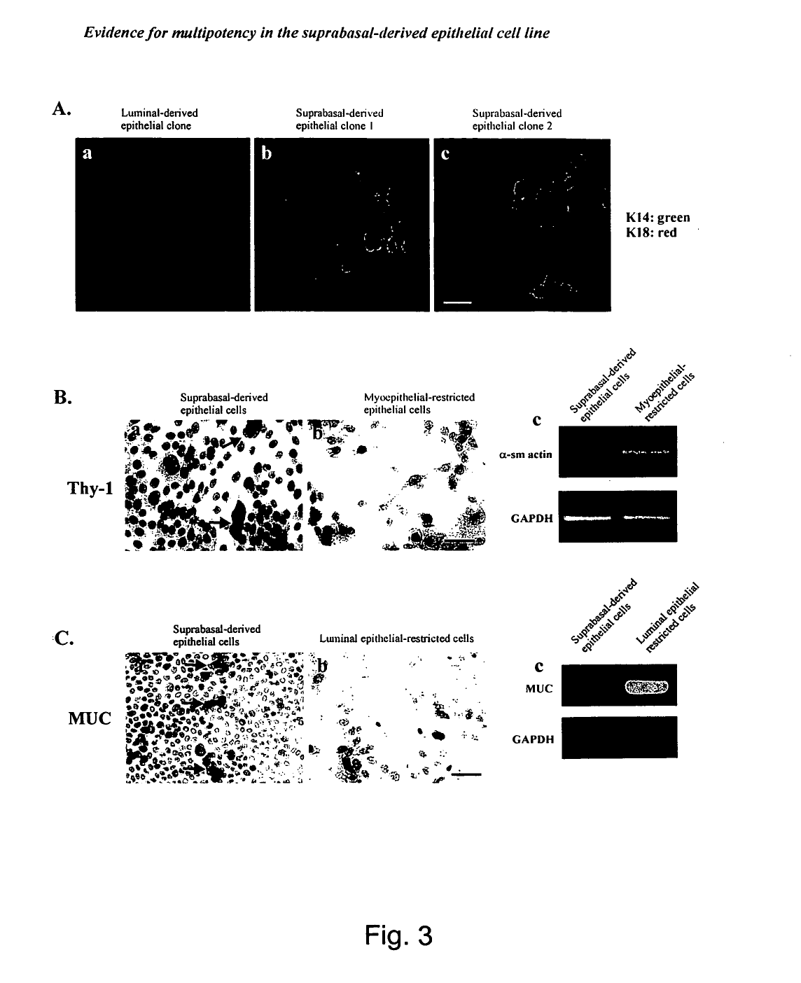

In example 3 this hypothesis was further strenghtened by double-staining clonal cultures for keratin K18 (luminal marker) and K14 (myoepithelial marker). From these experiments it was clear that whereas the luminal-derived epithelial cell line did not generate any myoepithelial cells, the suprabasal-derived cell line readily formed mixed clones of luminal epithelial and myoepithelial cells. This conclusion was further emphasised by the observation that the myoepithelial cells represented a primitive level of myoepithelial differentiation because <1% of the cells expressed other myoepithelial markers such as Thy-1 and furthermore that such Thy-1 expressing myoepithelial-like cells also expressed α-smooth muscle actin which is restricted to postmitotic myoepithelial cells in vivo (FIG. 3A, b, c) (Sapino et al. 1990).

In example 3 data are also presented showing that the K18⁺ cells were also precursor cells of the lineage-restricted progeny within the luminal compartment. It is shown that these cells could further mature within this compartment to differentiated sialomucin-expressing cells. Taken together, these observations provide evidence for the existence of a suprabasal, at least bi-potent epithelial cell belonging to the luminal epithelial lineage that can give rise to differentiated myoepithelial and luminal epithelial cells, and their precursors.

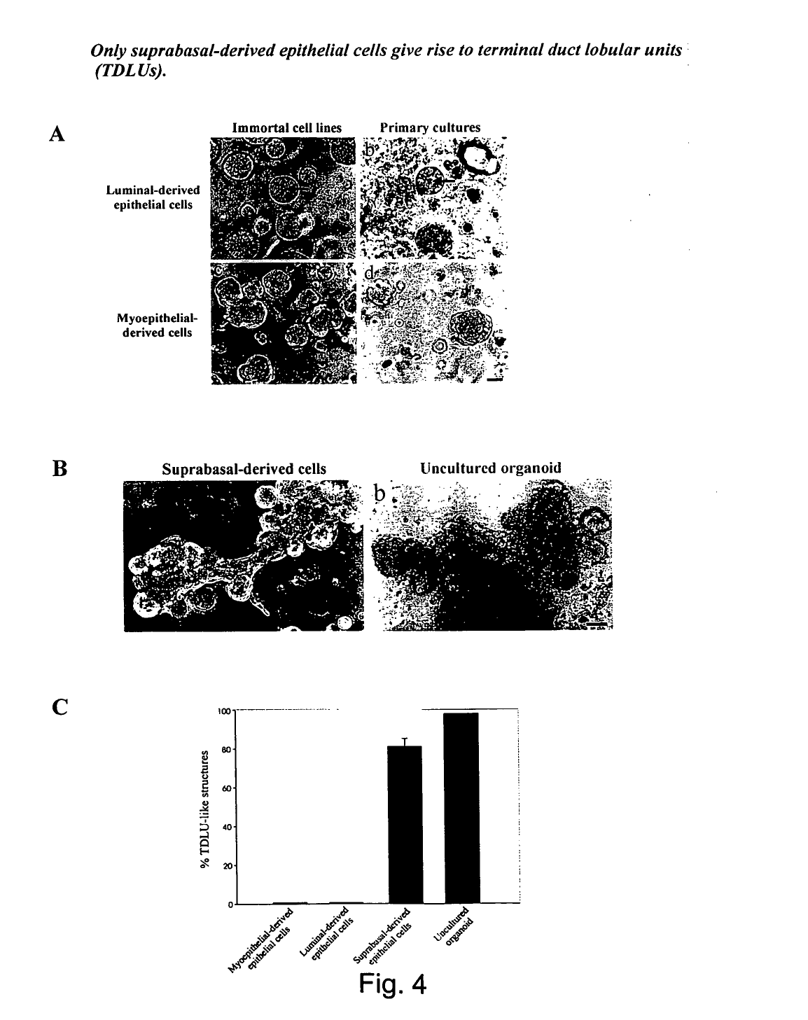

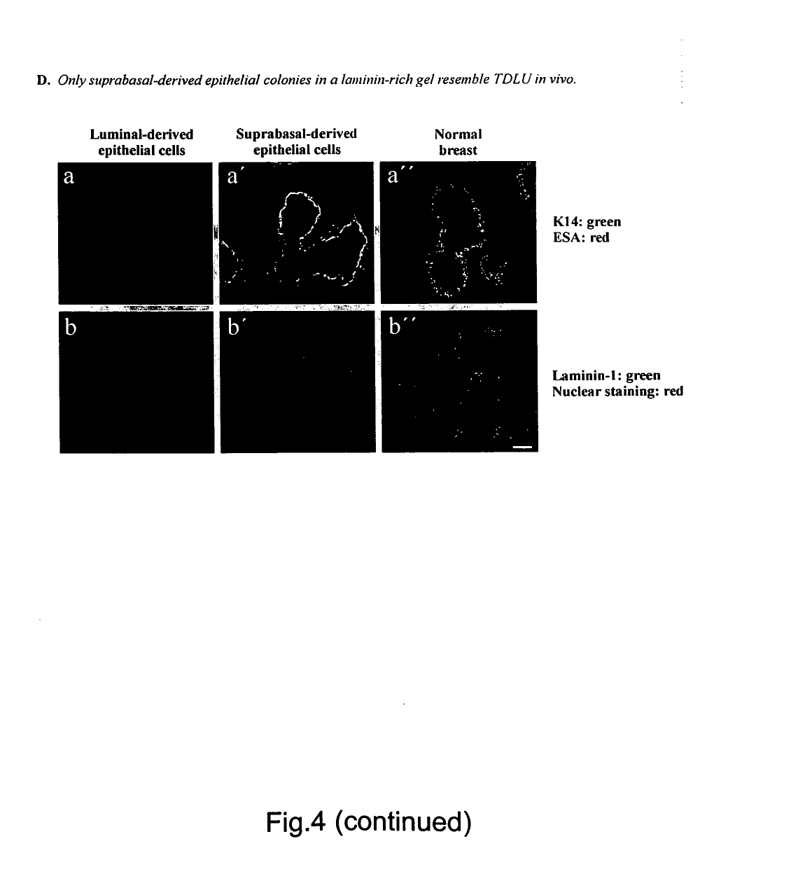

The perhaps most striking feature of the cells of the invention is the demonstration in example 4 that an immortalised suprabasal-derived epithelial cell line in culture is capable of forming branching structures resembling terminal duct lobular units of the mammary gland not only by marker expression as demonstrated in example 3 but also in morphology. In general, the criterion for stem cells of the breast is their ability to regenerate the entire structure of the mammary gland. In mice and rats the ability to regenerate the entire structure of the mammary gland upon reimplantation of cells in syngeneic gland-free fat pads has been adapted as the standard criteria for stem cells (Smith and Medina 1988). In example 4 a similar test in a laminin-rich gel was performed. When suprabasal-derived epithelial ESA+/MUC− cells were embedded into the gel and cultured under these three-dimensional culture conditions they gave rise to formation of larger, elaborate morphologies resembling the entire functional unit of the breast gland, i.e. the terminal duct lobular unit (TDLU). In this respect the suprabasal-derived epithelial cell-lines formed TDLU-like structures at a frequency (73%) similar to that recorded for suprabasal cells that were freshly prepared from primary cultures from two different biopsies. The TDLU-like structures were stained for differentiation markers of normal breast and revealed a remarkable similarity to TDLU's in vivo (FIG. 4D). Importantly, this was true for all the three cell lines (D492, D490 and TH69). The present inventors conclude that the suprabasal-derived at least bi-potent cell lines posses many of the characteristics of a human progenitor cell of the breast gland, and accordingly a preferred embodiment of the invention is an immortalised cell line that in culture is capable of forming branching structures resembling terminal duct lobular units of the mammary gland in morphology and/or by marker expression.

As demonstrated in example 5 cells isolated, immortalised and established as a cell line according to the methods disclosed in the present invention comprises cells that are positively staining for the keratin K19. It is considered a major novel and surprising conclusion that the progenitor cells of the human breast reside in a keratin K19⁺ compartment since it has been the technical prejudice within the field that the potential stem cells of the breast were keratin K19⁻ cells. This was based on earlier observations that benign proliferative lesions and highly proliferative breast cell lines were keratin K19⁻, and that keratin K19⁺ cells proliferate poorly in culture (Rønnov-Jessen et al. 1996). However, more recent evidence supports our observation of a keratin K19⁺ precursor cell compartment. First, keratin K19 is one of the earliest keratins expressed in the embryo, and whereas the foetal breast contains a homogeneously keratin K19⁺ luminal epithelial compartment, keratin K19⁻ luminal cells arise only in adulthood (Anbazhagan et al. 1998). Second, more than 90% of breast carcinomas are K19⁺. While it could be argued that K19⁻ luminal epithelial cells could turn on K19 along with malignant transformation, so far all malignant or preneoplastic transformations of non-malignant K19⁻ breast cell lines have resulted in K19⁻ tumour cells (Petersen et al. 1998; Santner et al. 2001). Third, studies of other organs, including liver, pancreas, skin, testes, and prostate have revealed that the “stem cell compartment” express keratin K19 (Stosiek et al. 1990; Fridmacher et al. 1995; Michel et al. 1996; Bouwens 1998; Hudson et al. 2001). This does not imply that all keratin K19⁺ cells are stem cells, since for instance the entire basal layer of the skin is K19⁺ and this by far exceeds the expected number of stem cells. Therefore, in preferred embodiments, the immortalised cell line comprises cells that are positive staining for the keratin K19.

The most preferred embodiment of the present invention is a human suprabasal-derived cell line possessing many of the characteristics of a stem cells of the human breast gland and which is exemplified by the immortalised D492 cell line. This immortalised cell line is deposited in accordance with the provisions of the Budapest Treaty on the International Recognition of the Deposit of Microorganisms for the Purposes of Patent Procedure at Deutsche Sammlung von Mikroorganismen und Zellkulturen GmbH (DSMZ) and has obtained the accession number DSM ACC 2529. Other embodiments of the present invention is an immortalised cell line that is capable of proliferating and capable of differentiating into cells of mammary gland luminal epithelial and myoepithelial cell lineages, and is derived from a cell selected from the group consisting of a rodent cell, a porcine cell, a ruminant cell, a bovine cell, a caprine cell, a equine cell, a canine cell, a ovine cell, a feline cell and a primate cell. In particular, an immortalised cell line that is selected from the group consisting of cells from mice, rats and rabbits is an important embodiment of the present invention, since it opens for new experimental possibilities e.g. transplantation experiments in syngeneic individuals, which is not practicable in the case of human cells. However, an immortalised cell line that is a human cell line is a preferred embodiment.

The isolation and establishment of three immortalised cell lines (D492, D490 and TH69) and two non-immortalised suprabasal luminal epithelial cell cultures (TH82 and TH95) show that the method for isolation of an at least bi-potent mammary gland tissue cell which is described in example 2 is a general method for the isolation of a bi-potent cell type sharing many of the characteristics of a stem cell of the breast gland. The method for isolation of an at least bi-potent mammary gland tissue cell, comprising the steps of: (i) separating of the breast tissue into two or more different cell types; (ii) culturing each of said different cell types under cell differentiation conditions and; (iii) selecting the cell type(s) that is/are capable of differentiating into at least two morphologically and/or phenotypically different cell types. In one specific embodiment of the method according to the invention, an at least bi-potent cell which is derived from luminal epithelial cells of a mammary gland and capable of proliferating and capable of differentiating into cells of mammary gland luminal epithelial and myoepithelial cell lineages is isolated.

One preferred method separating the breast tissue cells into two or more different cell types is to disaggregate the tissue mechanically followed by an enzymatically disaggregation with collagenase, as described in example 2.

Example 2 describes a preferred embodiment of the subsequent culture of disaggregated breast tissue. The culture is performed by placing the organoids in collagen-coated culture flasks added CDM3 (Petersen and van Deurs, 1987) culture medium, and kept 37° C. in a standard cell incubator in a humidified atmosphere of 75% N₂, 20% O₂, and 5% CO₂. In the experiment reported in example 2 the culture flasks were coated with 8 μg/cm² collagen. While this collagen coating results in a surface which is optimal for the plating of primary human breast epithelial cells, other coatings may be applied for non-human cells. For instance coating with fetal calf serum has been reported to facilitate plating of primary epithelial cells. Also the exact amount of collagen may vary such as between 0.1-100 μg/cm², 0.5-50 μg/cm², 1-30 μg/cm², 3-20 μg/cm², 5-15 μg/cm² or 6-10 μg/cm² and depend on the exact type and stock. Whereas the CDM3 medium is preferred it is contemplated that almost identical cell cultures may be obtained with other media. One example is the DMEM/F-12 medium 1:1 supplemented with 2 mM glutamine and a number of growth factors, see table 3.

Both in the case of isolation of luminal epithelial cells and suprabasal-derived epithelial cells the organoids was cultured with a change of medium three times a week. When the organoids had spread out to monolayers in primary culture cells were trypsinised and filtered as described by (Péchoux et al. 1999) and all cell separations were carried out by use of specific antibodies coupled to the matrix of the MiniMACS magnetic cell separation system according to the manufacturer's instructions (Militenyi Biotech, Gladbach, GmBH). Within the concept of this invention is also the use of other systems for cell separation which is based on a stepwise selection or enrichment of cells which express no or low levels of sialomucin and relative high levels of epithelial specific antigen (ESA). One such cell separation method is flow cytometry (Stingl et al. 1998).

Isolating an at least bi-potent suprabasal positioned luminal epithelia cell of the breast by any such sequential procedure involving culture of primary mamma organoids followed by a immunological based enrichment of ESA⁺/MUC⁻ cells and resulting in a cell which is capable of forming a cell culture comprising cells which are positive staining for the luminal epithelial marker ESA (ESA⁺) and negative or weakly positive staining for sialomucin (MUC⁻), (ESA⁺/MUC⁻) cells are stipulated to be within the scope of present invention.

Such isolated cells have many important industrial uses. In one embodiment of the invention the cells are used to establish a method for testing the toxic effect, if any, of a substance on mammary gland epithelial cells. The method comprising: (i) culturing or maintaining the cells in a non-toxic medium; (ii) adding the substance to be tested to the medium; and (iii) determining the response, if any, of the cells, including changes in cell growth rate, cell death rate, apoptosis, cell metabolism, inter- as well as intra-cellular communication, adhesion, morphology, mRNA or protein expression and antigen expression.

The term “growth rate” is defined as the net increase in cell number within a given time period.

The term “apoptosis” is used to describe the normal cellular process involving an active, genetically programmed series of events leading to the death of a cell. Often toxic substances induce apoptosis in cells. In contrast to apoptosis, which is, a basic physiological process necrosis describes the accidental cell death that is the cell's response to a variety of harmful conditions and toxic substances.

The term “cell death rate” is defined as the number of cells dying per unit time. The cell death rate is determined by the rate of which cells undergo apoptosis or necrosis.

The term “intercellular communication” is defined as signalling between individual cells as elicited by cytokines, extracellular matrix components, adhesive molecules or the like.

The term “intracellular communication” is defined as the signalling within an individual cell typically elicited by activation of membrane bound or nuclear receptors or other cell signalling mediators.

The cells of the present invention are keratin K19-expressing, suprabasally located cells within the luminal epithelial lineage that are putative precursor cells of terminal duct lobular units in the human breast. Since it long has been assumed that human breast cancer originates from the luminal epithelial lineage within the terminal duct lobular units (TDLU), and since more than 90% of human breast carcinomas are keratin K19 positive, it appears that the cells of this invention are identical to the cellular origin of most human breast cancers, and therefore constitute a very attractive cellular model system for breast cancer. Thus an even more preferred embodiment of the invention is a method for testing the carcinogenic effect, if any, of a substance on mammary gland epithelial cells, the method comprising: (i) culturing the cells of the invention in a growth medium which maintains the cells as non-transformed cells; (ii) adding the agent, compound or factor under test to the cell culture; and (iii) determining the neoplastic response, if any, of the so contacted cells by changes in morphology, tumorigenicity in animals, mRNA expression and/or antigen expression as well as other changes associated with carcinogenesisi.

By the term “neoplastic response” the present inventors here refer to tumor formation as a consequence of clonal expansion of genetically altered cells.

By the term “tumorigenicity in animals” the present inventors here refer to the important aspect of neoplastic cells to form tumours in animals. The present invention refers to a tumorigenicity test performed on either syngeneic or immuno-incompetent animals. However in a preferred embodiment the tumorigenicity test comprise the introduction of said treated cells into an immune incompetent test animal.

By the term “carcinogenicy” the present inventors here refer to tumor formation related to the effect of tumor promoting or genotoxic exposures such as exposure to radiation, tar and various carcinogenic substances.

Since the cells of the invention are characterised by being at least bi-potent cells with stem cell characteristics the cells can be exploited to test the ability, if any, of a substance to modulate the differentiation of non-terminal differentiated mammary gland epithelial cells. Thus one aspect of the invention relates to a method of testing a substance for its ability to modulate the differentiation of mammary gland epithelial cells comprising: (i) culturing or maintaining the cells of the present invention in a medium which in itself does not modulate the differentiation; (ii) adding the substance under test to the cell culture; and (iii) determining the differentiation modulation responses, if any, of the so contacted cells by changes in cell growth rate, cell death rate, apoptosis, cell metabolism, inter- as well as intra-cellular communication, morphology, mRNA or protein expression or antigen expression as well as other changes which is associated with differentiation.

An interesting aspect of the invention relates to the use of the cells according to the invention to test substances for their ability to interact with a particular cellular protein.

To ensure expression of the specific protein in question a nucleic acid fragment which includes a nucleic acid sequence encoding for the specific protein may be obtained, inserted into a suitable expression vector and the resulting “gene construct” transfected into any of the cells or cell lines of the present invention.

The term “expression vector” is used to denote a DNA molecule, linear or circular, which comprises a segment encoding a polypeptide of interest operably, linked to additional segments that provide for its expression. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both. Many such “expression vectors” are described in the art. A comprehensive survey of such expression vectors can be found in Ausubel et al. 2000, which is incorporated herein by reference.

The gene construct may be introduced into the host of this invention cell by transduction or transfection. Detailed description of useful methods may be found in Ausubel et al. 2000, and Sambrook, et al. 1989, both of which is incorporated herein by reference.

The next step in the test is to add the substance to be tested to the cells; and finally to determine the interaction, if any, with the cellular protein. Such interaction may be deduced from changes in the phenotype, e.g. by changes in cell growth rate, cell death rate, apoptosis, cell metabolism, inter- as well as intra-cellular communication, morphology, mRNA or protein expression, antigen expression or other changes which either directly or indirectly is supposed to be associated with said protein.

Since the growth of breast epithelial cells in vivo is guided by hormones such as estrogen and progesterone a particular important variation of this embodiment is a method for the detection of interaction between a cellular protein and a given substance in which said cellular protein is selected from the group consisting of cellular receptors in particular estrogen receptor-alpha, estrogen receptor-beta and progesterone receptor.

In the present context the term “receptor” denotes a cell-associated protein that binds to a bioactive molecule (i.e., a ligand) and mediates the effect of the ligand on the cell. Typically the binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

One particular intriguing embodiment of the present invention is to transplant a vertebrate host with a cell according to present invention. Such cells could be transfected with a heterologous nucleotide sequence and consequently express such heterologous protein. The term a “heterologous nucleotide sequence” is herein used to describe a DNA sequence inserted within or connected to another DNA sequence which codes for polypeptides not coded for in nature by the DNA sequence to which it is joined. The term a “heterologous protein” is herein used to describe a polypeptide that is being expressed in a context not found in the non-transfected cells. Consequently, it is within the scope of the present invention to administer a given protein or gene of interest to an individual in need thereof, comprising the step of transfecting the cell-population of said at least bi-potent luminal derived epithelial cells of a mammary gland with a vector comprising DNA or RNA which expresses the protein or gene of interest and introducing the transfected cell into said individual. Thus the use an such at least bi-potent cell to prevent and/or treat cellular debilitations, derangements and/or dysfunctions and/or other disease states in mammals, comprising administering to a mammal a therapeutically effective amount of said cells, or cells or tissues derived therefrom is within the scope of the present invention.

Since the at least bi-potent immortalised suprabasal-derived epithelial cell line of the present invention is capable of forming branching structures resembling terminal duct lobular units of the mammary gland not only by marker expression but also in morphology it is contemplated that such cells can be used for tissue repair or transplantation. For instance if patients suffer from drastic mastectomies, it is possible to isolate at least bi-potent mammary gland tissue cells from their breast by the methods of the present invention. Further it is possible to immortalise said cells, and use them for reimplantation to re-engineer a breast tissue in situ. In a further useful embodiment of the invention, a method of tissue repair or transplantation in mammals, comprising administering to a mammal a therapeutically effective amount of an at least bi-potent luminal derived epithelial cells of a mammary gland, or cells or tissues derived therefrom is contemplated.

Adult stem-like cells of the breast could conceivably also be used for derivation of other tissues. While this technology has not yet been demonstrated, the chances are that the present inventors may be able to derive both hair forming cells and skin tissues from these cells because breast cells share an embryonic origin with these cells.

In a still further aspect, the invention pertains to the use of said at least bi-potent luminal derived epithelial cells of a mammary gland for the formulation of a pharmaceutical composition comprising: a therapeutically effective amount of said cells, or cells or tissues derived therefrom; and a pharmaceutically acceptable carrier. To complement the pharmaceutical composition, said composition could further comprise a proliferation factor or lineage commitment factor.

In a final aspect of the invention said at least bi-potent luminal derived epithelial cells of a mammary gland is used to produce a diagnostic agent comprising said cells, or any part thereof. One such diagnostic agent could be a specific antibody directed against an antigen specific for said at least bi-potent cell of a mammary gland.

Part of the invention described in the present application has, after the filing of the priority application of the present application, led to the publication Gudjonsson, T., Villadsen, R., Nielsen, H. L., Rønnov-Jessen, L., Bissell, M. J., Petersen, O. W. 2002. Isolation, immortalization, and characterization of a human breast epithelial cell line with stem cell properties. Genes Dev. 16:693-706 published on 15 Mar. 2002.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

### LEGENDS TO FIGURES

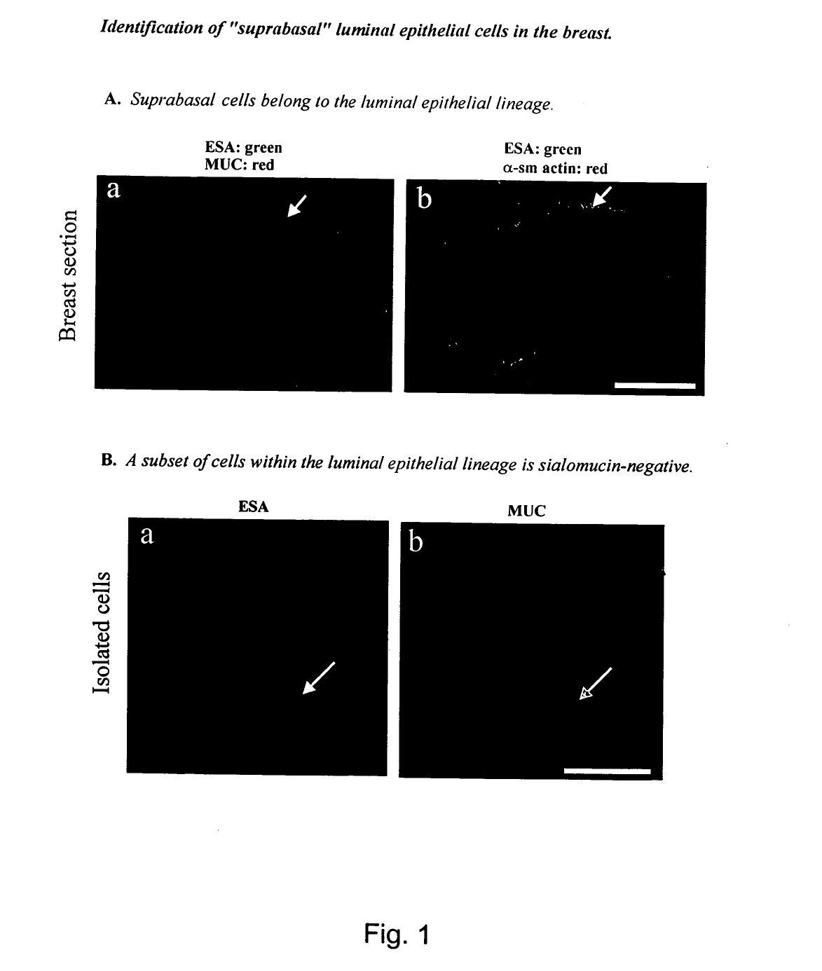

FIG. 1. Identification of “suprabasal” luminal epithelial cells in the breast.
A. Suprabasal cells belong to the luminal epithelial lineage. (a) Double labelling immunofluorescence staining of epithelial specific antigen (ESA) and sialomucin (MUC1). The arrow indicates an example of an epithelial cell which apparently does not reach the lumen and fails to express sialomucin. (b) Double labelling immunofluorescence staining of ESA and α-smooth muscle actin. Note that the suprabasal epithelial cells (arrow) are resting on a basal layer of α-smooth muscle actin-positive myoepithelial cells. (x1100; bar=20 μm).
B. A subset of cells within the luminal epithelial lineage is sialomucin-negative. Uncultured, trypsinized breast epithelial cells were double stained to demonstrate ESA (green) and sialomucin (red). Whereas the majority of cells were double-stained for ESA and sialomucin, a small fraction stained for ESA only (arrow). (x1100; bar=20 μm).

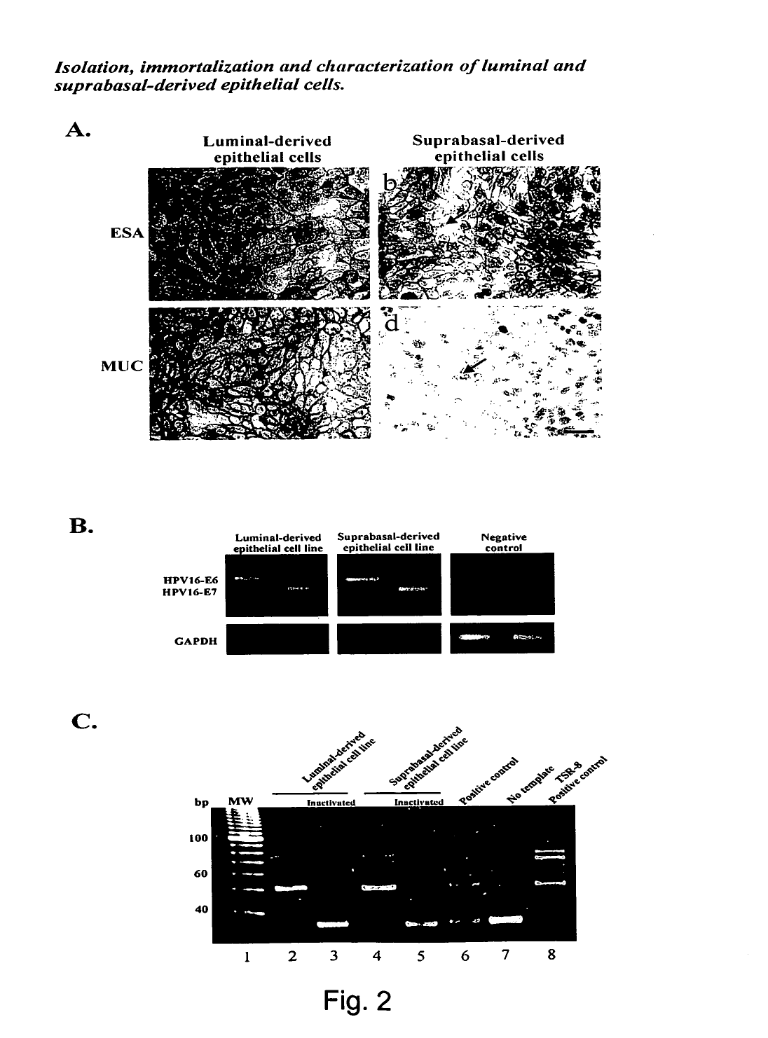

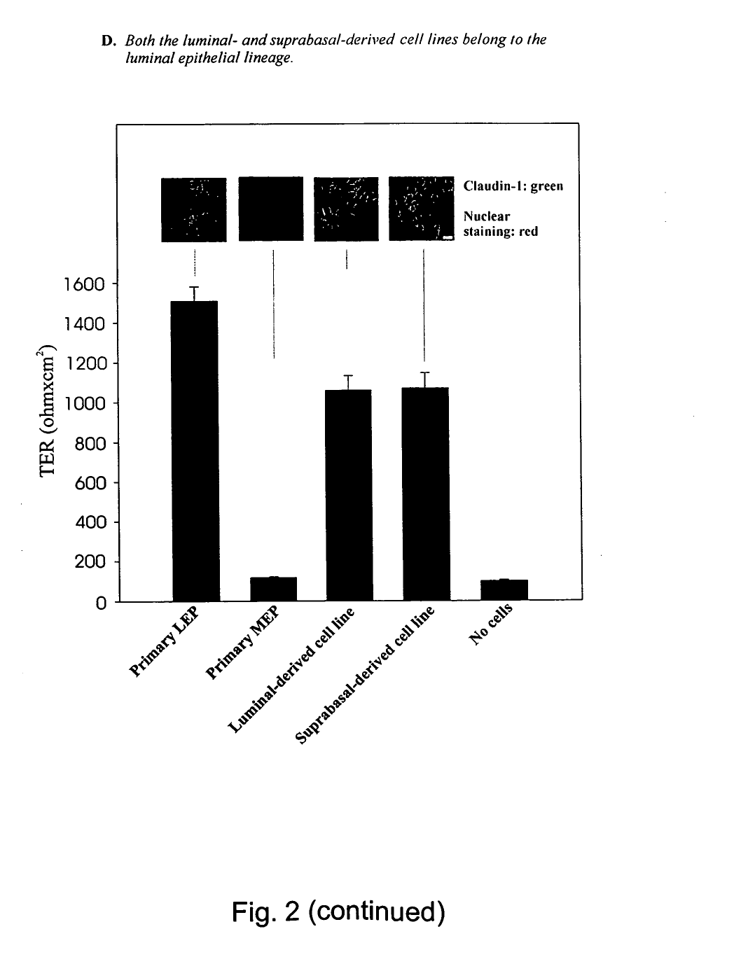

FIG. 2. Isolation, immortalisation and characterization of luminal and suprabasal-derived epithelial cells.
A. The luminal and suprabasal-derived epithelial cells differ by sialomucin expression. Immunoperoxidase staining with ESA (upper row) and MUC1 (lower row) of the luminal (left column) and the suprabasal-derived epithelial cell line (right column). Note that whereas the luminal epithelial-derived cell line is homogenous in its staining pattern and expressed both ESA and sialomucin (MUC1), the suprabasal epithelial cell line is heterogeneous (arrows) and essentially negative for sialomucin. (x250; bar=50 μm).
B. The cell lines express E6 and E7 stably. RT-PCR of HPV16 E6 and E7 show that both the luminal and the suprabasal-derived epithelial cells are stably transduced as compared to a negative control.
C. The cell lines exhibit telomerase activity. TRAP assay of equal numbers of luminal and suprabasal-derived epithelial cell lines showed telomerase activity in transduced cells. Lane 1: molecular weight markers, lane 2: luminal-derived epithelial cell line, lane 4: suprabasal-derived epithelial cell line, lane 3 and 5: heat inactivated negative control of the cell lines, lane 6: positive control pellet, lane 7: negative control without cell lysate, lane 8: positive control TSR8 control template.
D. Both the luminal and suprabasal-derived epithelial cell lines belong to the luminal epithelial lineage. Confluent cultures were plated on Transwell filters and assayed for transepithelial resistance (TER), and parallel monolayer cultures were double-stained for claudin 1 as well as propidium iodide to visualize the nuclei (inserts). Primary luminal epithelial cells (LEP) and myoepithelial cells (MEP) are readily discriminated by TER and claudin-i expression. The luminal and suprabasal-derived cell lines resemble primary luminal epithelial cells by a high TER and a prominent expression of claudin-1. (x400; bar=20 μm).

FIG. 3. Evidence for multipotency in the suprabasal-derived epithelial cell line.
A. Doublestaining with luminal epithelial K18 and myoepithelial K14 in clones of the luminal-derived cell line and the suprabasal-derived cell line. Clonal cultures of the luminal-derived (a) and the suprabasal-derived epithelial cells (b, c) were double-stained with keratin K18 and K14. No evidence for myoepithelial cells, i.e. no K14-staining was found in any of the luminal-derived clones. Conversely, a mixture of K18- and K14-positive cells was frequently present in the suprabasal-derived epithelial clones. (x200; bar=40 μm).
B. Evidence of spontaneous maturation to myoepithelial-restricted cells. Immunoperoxidase staining of Thy-1, a marker for myoepithelial cells, in cultures of suprabasal-derived cells before (a) and after (b) purification in a Thy-1 retaining column. The spontaneous occurrence of Thy-1 stained cells is limited to less than 1% (arrows). However, upon purification, a myoepithelial restricted subline can be obtained which also express α-smooth muscle actin as shown by RT-PCR(c). (x250; bar=50 μm).
C. Evidence for maturation to luminal epithelial-restricted cells. Suprabasal-derived epithelial cells were cleared of sialomucin-positive cells and stained for sialomucin after 2 weeks (a, arrows), and after further retention of the newly formed sialomucin-positive cells (b). The MUC stainings was confirmed by RT-PCR (c). (x250; bar=50 μm).

FIG. 4. Only suprabasal-derived epithelial cells give rise to terminal duct lobular units (TDLU).
A. Luminal epithelial- and myoepithelial-derived cells make colonies with distinct morphologies in a laminin-rich gel. Immortalized (a, c) and primary (b, d) luminal epithelial-derived cells (a, b) and myoepithelial-derived cells (c, d) were embedded as single cells in a laminin rich gel. Both immortal luminal epithelial- and myoepithelial-derived cells resembled the corresponding primary cells. Whereas the luminal epithelial cells formed acinus-like spheres with a central lumen, the myoepithelial cells formed irregular solid clusters of cells. (x200; bar=25 μm)
B. Suprabasal-derived epithelial cells make an elaborate TDLU-like structure in a laminin-rich gel. Suprabasal-derived epithelial cells (a) were embedded as single cells in a laminin-rich gel and compared with the morphology of freshly isolated, uncultured TDLU organoids (b). Both consist of small branching ductules terminating in globular acinus-like structures. (x100; bar=50 μm)
C. Quantitation of TDLU-like structures in laminin-rich gels. Luminal and suprabasal-derived epithelial cells and myoepithelial-derived cells were embedded inside laminin-rich gels and allowed to grow for 12 days. The number of TDLU-like structures was quantified by phase contrast microscopy.
D. Only suprabasal-derived epithelial colonies in laminin-rich gels resemble TDLU in vivo. Sections of laminin-rich gels containing luminal-derived (left column) and basal-derived epithelial cells (middle column) were compared with sections of normal breast tissue (right column) and doublestained for ESA and keratin K14 (a-c), and propidium iodide and laminin-1 (d-f). Only the suprabasal-derived epithelial cells showed a differentiation pattern reminiscent of normal breast tissue with an inner layer of ESA-positive cells and an outer layer of K14-positive/ESA-negative cells and basal deposition of a laminin-1 containing basement membrane (x400; bar=151 μm).

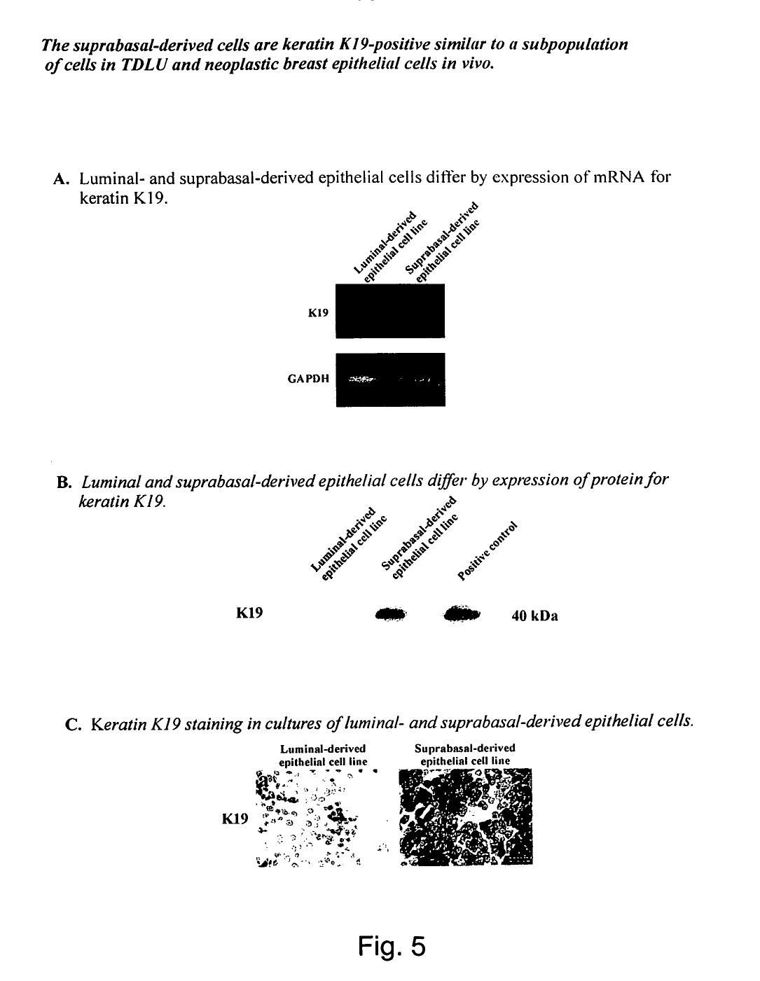

FIG. 5. The suprabasal-derived cells are keratin K19-positive similar to a subpopulation of cells in TDLU and neoplastic breast epithelial cells in vivo.
A. Luminal epithelial- and suprabasal-derived epithelial cells differ by expression of mRNA for keratin K19. RT-PCR of keratin K19 in luminal epithelial- and suprabasal-derived epithelial cells with expression in the suprabasal-derived cells only.
B. Luminal epithelial- and suprabasal-derived epithelial cells differ by expression of keratin K19. Immunoblot of keratin K19 of protein lysates from luminal epithelial- and suprabasal-epithelial derived cells showing expression in the suprabasal-derived cells only.
C. Keratin K19 staining in cultures of luminal- and suprabasal-derived epithelial cells. Cultures were stained for keratin K19 by immunoperoxidase and counterstained with hematoxylin. Whereas the luminal-derived epithelial cells were completely negative, the other cell line was heterogeneous with a large contribution from keratin K19-positive cells. (x250; bar=50 μm).

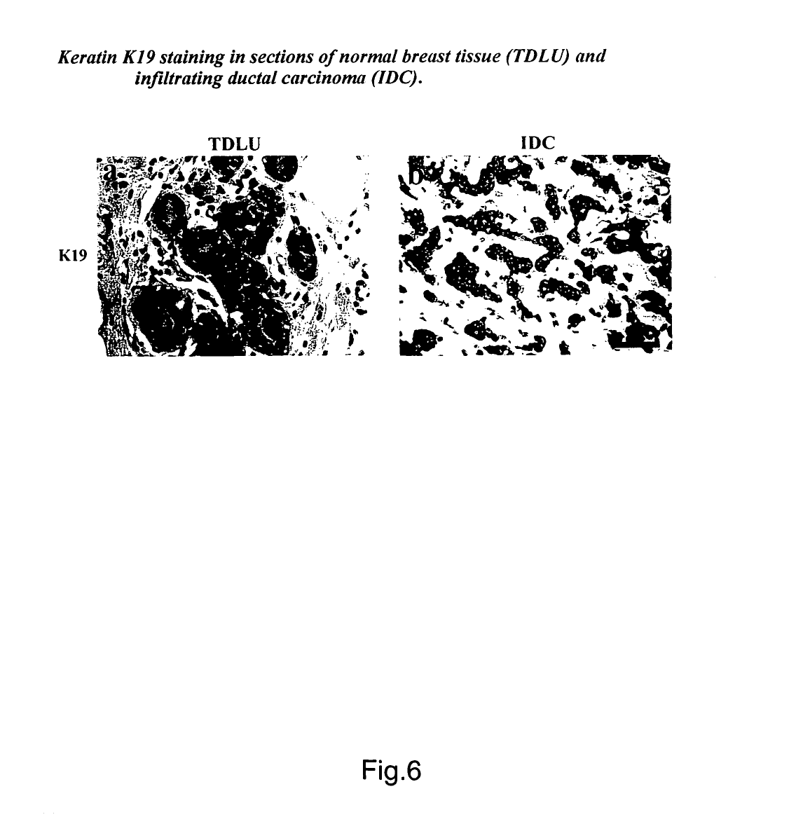

FIG. 6. Keratin K19 staining in TDLU and infiltrating ductal carcinoma (IDC).
Section of breast tissue showing TDLU (a) and IDC (b) stained for keratin K19 and counterstained with hematoxylin. Note the heterogeneous staining and the presence of several stained suprabasal cells in the TDLU and the more widespread staining of neoplastic epithelial cells in the carcinoma. (x250; bar=50 μm).

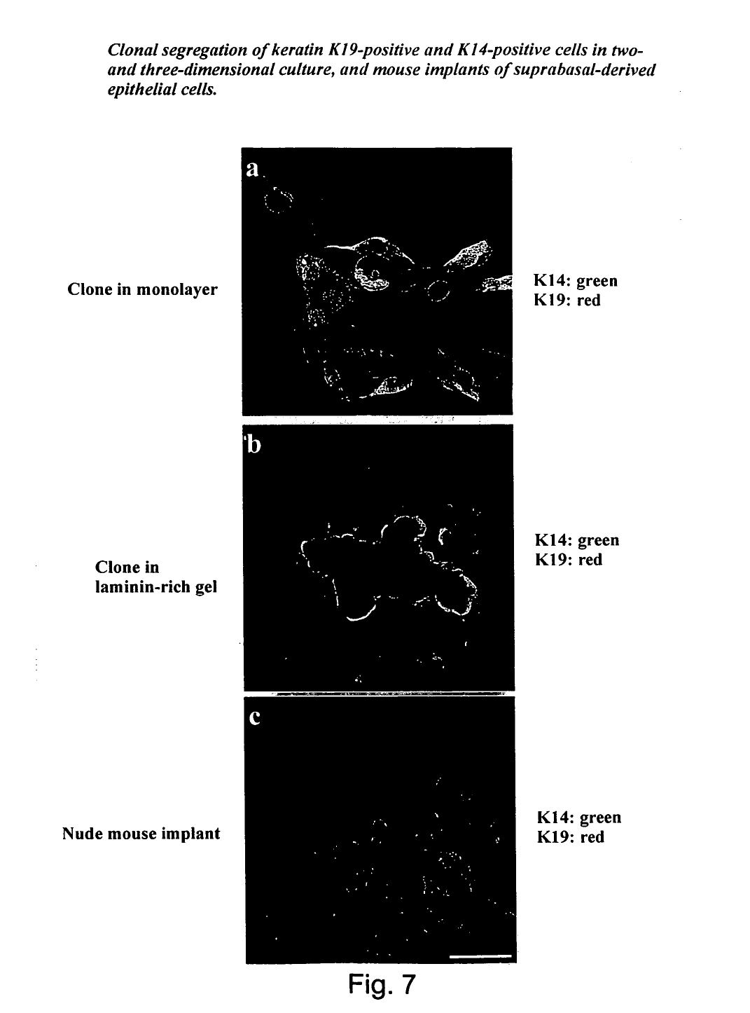

FIG. 7. Clonal segregation of keratin K19-positive and K14-positive cells in two- and three-dimensional culture and mouse implants of suprabasal-derived cells.
Clonal culture of suprabasal-derived cells on monolayer collagen-coated plastic (A), in a laminin-rich gel (B) and implanted orthotopically in the nude mouse (C) double-labelled with keratin K19 and keratin K14. The monolayer cultures show distinct evidence of bi-potency, and in three-dimension in culture as well as in vivo this organizes further into TDLU-like structures including terminal ducts and acini (x400; 40 μm).

### EXAMPLES

### Example 1

Identification of “Suprabasal” Luminal Epithelial Cells in the Breast

In culture, a putative “stem” cell of the human breast was defined based on a positive staining for the luminal epithelial marker ESA and a negative or weakly positive staining for sialomucin (MUC) (Stingl et al. 1998). To investigate if such a candidate stem cell could be identified in human breast in vivo, the present inventors double-stained histological sections of normal human breast tissue with epithelial-specific antigen (ESA) and sialomucin (MUC).

In general, immunocytochemistry and confocal microscopy was performed as follows. Normal human breast tissue was obtained as biopsies from patients undergoing reduction mammoplasty for cosmetic reasons. The use of human material has been reviewed by the Regional Scientific-Ethical Committees for Copenhagen and Frederiksberg, Denmark and approved with reference to (KF) 01-161/98. The tissue was frozen in n-hexan (Merck, Darmstadt, Germany) and mounted in Tissue Freezing Medium™ (Leica Instruments, Heidelberg, GmbH) for sectioning. Frozen tissue was sectioned at an 8-μm setting in a cryostat. The sections and cell cultures were dried for 15 min at room temperature and fixed in methanol as previously described (Petersen and van Deurs 1988). Primary antibodies directed against sialomucin (MAM6, clone 115D8, Biogenesis Ltd., Poole, UK), epithelial-specific antigen (ESA; VU-1D9, NovoCastra, Newcastle upon Tyne, UK ) and α-smooth muscle actin (1A4, Sigma-Aldrich, Vallensbœk, Denmark) were used. For an overview of the primary antibodies used throughout the study, see Table 4. Rabbit anti-mouse immunoglobulins (Z259, DAKO, Glostrup, Denmark) were used as secondary antibodies and a peroxidase conjugated anti-peroxidase mouse mAb was used as tertiary antibody (P850, DAKO, Glostrup, Denmark). The peroxidase reactions were performed using 0.5 mg/ml 3,3-diaminobenzidine (Sigma-Aldrich, Vallensbœk, Denmark) and 0.5 μl/ml of 30% H₂O₂ (Merck, Darmstadt, Germany, purchased from Struers KEBO LAb A/S, Albertslund, Denmark) for 10 minutes. The sections were counterstained with hematoxylin (Mayer's hematoxylin, MHS-16, Sigma-Aldrich, Vallensbœk, Denmark). For double-labeling experiments the present inventors used iso-type specific antibodies, all from Southern Biotechnology (Southern Biotechnology Associates, Birmingham, Ala.) as previously described (Rønnov-Jessen et al. 1995). Antibody incubations were carried out for 30 min, and specimens were rinsed twice for 5 min each, all at room temperature. Some sections received a nuclear counter stain with 1 μg/ml propidium iodide (Molecular Probes, Eugene, Oreg.). Afterwards sections were mounted with coverslips by use of Fluoromount-G (Southern Biotechnology Associates, Birmingham, Ala.) supplemented with 2.5 mg/ml n-propyl gallate (Sigma-Aldrich, Vallensbœk, Denmark) as previously described (Rønnov-Jessen et al. 1992). Immunofluorescence was visualized using a Zeiss LSM 510 laser scanning microscope (Carl Zeiss, Jena, GmbH). Sections were observed by use of a 20×, 40×, 63× or 100× objective and sliced in the z-plane into 0.25 μm-thick focal planes and exposed to visualize FITC and Texas red or propidium iodide.

Most luminal epithelial cells stained as expected, i.e. MUC was expressed on the apical surface and ESA at the basolateral surface (FIG. 1A).

However, it was also evident that a subset of ESA⁺ cells in occasional acinar profiles was indeed abluminal in location with no visible extensions reaching the lumen (arrow, FIG. 1A, a). To confirm that these cells were distinct from myoepithelial cells, the present inventors double-stained for ESA and the myoepithelial marker α-smooth muscle actin (α-sm actin). As expected, myoepithelial cells were negative for ESA, while a very minor population of basal cells were suprabasal and did not appear to reach the lumen or stain with α-sm actin (arrow, FIG. 1A, b). If these cells truly never reached the lumen—not even outside the sectioned plane—a sample of smeared trypsinized, uncultured breast cells should contain two ESA⁺ luminal epithelial populations: one major being MUC⁺ and a minor being MUC⁻. This was found to be the case as evidenced by doublestaining of such smears for ESA and MUC (FIG. 1B, a and b). The average frequency of MUC⁻ cells in such preparations was 8±3%.

From these experiments it is concluded that suprabasally positioned, abluminal cells within the luminal epithelial lineage also exist in the human breast.

### Example 2

Isolation, Immortalization and Characterization of Luminal and Suprabasal-Derived Epithelial Cells.

In order to show that the cells described in example 1 indeed have stem cell properties, the cells were isolated by immunomagnetic sorting and characterized.

Briefly, the luminal epithelial cells were purified from two consecutive sialomucin-columns and the suprabasal epithelial cells were purified as the flow-through from a sialomucin-column which was later retained in an ESA-column. To generate cell lines, the present inventors immortalised both populations with an E6/E7 construct of HPV16. The resulting established cell lines are referred to below as the luminal and suprabasal-derived epithelial cells, respectively.

Cell Culture

Breast luminal epithelial cells were generated from primary cultures of biopsies from patients undergoing reduction mammoplasty for cosmetic reasons. The tissue was prepared as previously described (Péchoux et al. 1999). Briefly, it was mechanically disaggregated followed by enzymatic disaggregation with collagenase (CLSIII, 900 units/ml, Worthington, purchased from Medinova, Hellerup, Denmark) to release epithelial organoids. The organoids were plated in CDM3 medium (Petersen and van Deurs 1987) on collagen-coated (8 μg/cm²; Vitrogen-100, Cohesion, Palo Alto, Calif.) T-25 flasks (Nunc, Roskilde, Denmark). Cells were kept at 37° C. in a Heraeus incubator in a humidified atmosphere of 75% N₂, 20% O₂, and 5% CO₂, and the medium was changed three times a week. In some instances organoids were trypsinized directly after collagenase digestion to obtain uncultured single cells for smears, which were fixed in methanol (Merck, Darmstadt, Germany) and further analysed.

Luminal Cells

Luminal epithelial cells were purified after organoids had spread out to monolayers in primary culture. Cells were trypsinized and filtered as previously described (Péchoux et al. 1999). All cell separations were carried out by use of the MiniMACS magnetic cell separation system according to the manufacturer's instructions (Militenyi Biotech, Gladbach, GmBH). The luminal epithelial cells were separated immunomagnetically from myoepithelial cells by retention in two consecutive anti-sialomucin (MAM6, clone 115D8, Biogenesis Ltd., Poole, UK) columns, and plated in CDM6 as peviously described (Péchoux et al. 1999). The cells were immortalised in passage 3 (for procedure, please see below), and cultured in CDM3 (Petersen and van Deurs 1987) until passage 11, where the medium was switched to H14 (Blaschke et al. 1994)(D382, Table III).

Suprabasal-Derived Epithelial Cells

Similar to the situation with the luminal epithelial cells suprabasal-derived epithelial cells were purified after organoids had spread out to monolayers in primary culture. Cells were similarly trypsinized and filtered and all cell separations were carried out by use of the MiniMACS magnetic cell separation system according to the manufacturer's instructions (Militenyi Biotech, Gladbach, GmBH).

The luminal cell population containing the suprabasal ESA⁺/MUC⁻ cells was isolated as the flow-through of an anti-sialomucin (MAM6, clone 115D8, Biogenesis Ltd., Poole, UK) column, and plated in Dulbecco's Modified Eagle Medium: Nutrient Mixture F-12 (DMEM/F-12, 1:1, GibcoBRL, LifeTechnologies, Tåstrup, Denmark) with glutamine (2 mM, GibcoBRL, LifeTechnologies, Tåstrup, Denmark) supplemented with cholera toxin (10 ng/ml, Sigma-Aldrich, Vallensbœk, Denmark), epidermal growth factor (100 ng/ml, PeproTech EC LTD, London, UK) and keratinocyte growth factor (10 μg/ml, PeproTech EC LTD, London, UK). Since all cultured breast epithelial cells in contrast to fibroblasts expressed β4-integrin (Gudjonsson et al. 2002a), residual fibroblasts were removed by retaining epithelial cells in a β4-integrin (3E1, Chemicon International, Temecula, Calif.) column. The purified epithelial cells, now in third passage, were then plated in DMEM/F-12 (1:1, GibcoBRL, LifeTechnologies, Tåstrup, Denmark) supplemented with glutamine (2 mM, GibcoBRL, LifeTechnologies, Tåstrup, Denmark), 10% fetal calf serum (E.C. approved, virus and mycoplasma tested, GibcoBRL, LifeTechnologies, Tåstrup, Denmark), insulin (3 μg/ml, Boehringer Mannheim, Roche, Hvidovre, Denmark), hydrocortisone (1.4×10⁶ M, Sigma-Aldrich, Vallensbœk, Denmark) and epidermal growth factor (100 ng/ml, PeproTech EC LTD, London, UK), and immortalised (please see below). In passage 6, the medium was switched to H14 (Blaschke et al. 1994).

D492

The suprabasal epithelial cells (D492, DMSZ no. DMS ACC 2529) were collected in passage 27 by retention of cells by an anti-ESA (VU-1D9, NovoCastra, Newcastle upon Tyne, UK) column (D492, Table III).

D490

The procedure was repeated with luminal cells from another biopsy with the following modifications: retention of β4-positive epithelial cells in second passage, flow-through of an anti-sialomucin column in passage 3, followed by immortalisation, switch to H14 in passage 6, and isolation of suprabasal epithelial cells, comparable to D492, by retention in an anti-ESA column in passage 10 (D490, Table III).

TH69

Finally, a third suprabasal cell line (TH69, Table III) was generated as the flow-through of an anti-sialomucin column, followed by retention in an anti-ESA column in second passage, immortalisation in passage 3, and subsequent switch to H14 in passage 6.

Non-Immortalised Suprabasal-Derived Epithelial Cells

To further verify that suprabasal cells with similar properties could be obtained without immortalisation, ESA⁺/MUC⁻ suprabasal cells were purified directly from primary cultures from two different biopsies as the flow-through of an anti-sialomucin (MAM6, clone 115D8, Biogenesis Ltd., Poole, UK) column followed by retention in an anti-ESA (VU-1D9, NovoCastra, Newcastle upon Tyne, UK) column (TH82 and TH95, Table III). The purified third passage suprabasal cells were embedded directly in 300 μl Matrigel® and cultured for 2 weeks in CDM3 medium (Petersen and van Deurs 1987), and within this period TDLU-like structures were formed.

Myoepithelial-Derived Cells

For isolation and purification of myoepithelial-derived cells the present inventors used an antibody against Thy-1 (AS02, Dianova, Hamburg, GmbH) (Gudjonsson et al. 2002a and b).

Establishment of Immortalized Cell Lines and Clonal Cultures

The luminal epithelial and suprabasal epithelial cells were transduced with sterile filtered retrovirus-containing supernatant from the PA317 LXSN HPV16E6E7 packaging cell line (CRL-2203, ATCC, Rockville, Md.) in the presence of 8 μg/ml polybrene (Sigma-Aldrich, Vallensbœk, Denmark) (Wazer et al. 1995). Transduced cells were selected in the presence of 100 μg/ml G418 (Life Technologies, Tåstrup, Denmark). Established cell lines were kept routinely in H14 medium (Blaschke et al. 1994). Clonal cultures were prepared according to a protocol for prostate epithelial cells (Hudson et al. 2000). Briefly, 1000 or 5000 cells (10 times more in 3-dimensional laminin rich gels) were plated onto collagen-coated (Vitrogen-100, Cohesion, Palo Alto, Calif.) T-25 flasks (Nunc, Roskilde, Denmark) in serum-free H14 medium and kept for 2 weeks prior to staining.

The resulting immortalised cell lines were tested for their tumorigenic properties in a standard tumorigenicity assay. Briefly, 10⁷ cells immortalised cells were inoculated subcutaneously into BALB/C nude mice. The mouse were followed by weekly inspections by palpation of the injection site. None of the cell lines were tumorigenic even after prolonged incubation.

Telomerase-Activity

The telomerase activity was determined with the TRAP assay using the TRAPeze Telomerase Detection Kit (Intergen, Oxford, UK) according to the manufacturer's instructions. Cells were grown to 70-80% confluence, trypsinized and counted. A lysate volume equal to 1000 cells was used for each reaction, and electrophoresed on a 12% nondenaturing acrylamide gel (BioRad, Herlev, Denmark), stained in SYBR green 1 (Molecular Probes, Leiden, The Netherlands) and visualized by UV transillumination and image recording in a Gel Doc 1000 (BioRad, Herlev, Denmark).

Transepithelial Electrical Resistance (TER)

For TER measurements, cells were plated on polycarbonate filters with a pore size of 0.41 μm (Corning Costar Corporation, Cambridge, Mass.) and allowed to reach confluency. A Millicell-ERS volt-ohm meter (Millipore, Hedehusene, Denmark) was used to determine the TER value. All TER values were normalized for the area of the filter and were obtained after background subtraction. All experiments were done in triplicate.

Results

As seen in FIG. 2A, the resulting established cell lines were ESA⁺/MUC⁺ (D382) and ESA⁺/MUC⁻ (D492), respectively. The cell lines displayed immortalised characteristics: they have been cultured for more than 50 passages over 2 years and continue to express both E6 and E7 (FIG. 2B), and a distinct telomerase activity which is absent from finite life span breast epithelial cells (FIG. 2C) (Stampfer et al 2001). Importantly, the immortalised D492 cell line were tested and found non-tumorigenic and in the case of the D492 cell line it was found that it has a diploid karyotype (46, XX) even after more than one year in culture and passages.

Three suprabasal cell lines with similar characteristics were established (D492, D490 and TH69). The latter was isolated as the flow-through of an anti-sialomucin column, directly followed by retention in an anti-ESA-column prior to immortalisation. Moreover, the retention step in a β4-integrin column was omitted. This indicates that the ESA⁺/MUC⁻ cells are indeed present initially and thus, do not occur as a result of immortalisation.

Whereas the luminal-derived epithelial cell line continued to be homogeneous (FIG. 2A, left), the suprabasal-derived epithelial cell line contained occasional subpopulations of ESA⁻ cells and MUC⁺ cells (FIG. 2A, right, arrow). Double immunofluorescence staining for ESA⁻ and MUC (not shown) revealed that this cell line contained three cellular subtypes: the majority population was ESA⁺/MUC⁻ and two minor populations were either ESA⁻/MUC⁻ or ESA⁺/MUC⁺. To substantiate that both cell lines belonged to the luminal epithelial lineage, even though one of them was essentially devoid of luminal epithelial MUC expression, the present inventors tested for a marker that is a hallmark of glandular epithelial phenotype—that of functional tight junctions. This was carried out by staining for the tight junction proteins claudin and occludin as described in example 1 but using antibodies against occludin (OC-3F10, Zymed Laboratories, San Francisco, Calif.) and polyclonal claudin-1 (Zymed Laboratories, San Francisco, Calif.) as primary antibodies, see also Table 4. Furthermore the level of transepithelial electrical resistance (TER) in confluent cultures on transwell filters was measured. By these criteria, primary luminal epithelial cells were readily distinguished from primary myoepithelial cells (FIG. 2D). As also seen in FIG. 2D, the newly established cell lines both resembled luminal epithelial cells by staining at the cell boundaries for tight junction proteins (see inserts) and exhibiting a high TER. The present inventors conclude that both cell lines belong to the luminal epithelial lineage.

### Example 3

Clonal Cell Lines of the Suprabasal-Derived Epithelial Cell Line are Multipotent

Clonal cultures were established and double-stained for keratin K18 (luminal marker) and K14 (myoepithelial marker) as described in example 1 by using antibodies against keratin K18 (F3006; Trichem Aps, Denmark) and keratin K14 (LL002, NovoCastra, Newcastle upon Tyne, UK) as primary antibodies, se also Table 4.

Whereas the luminal-derived epithelial cell line did not generate any myoepithelial cells and stained for K18 only, the suprabasal-derived cell line readily formed mixed clones of luminal epithelial and myoepithelial cells (FIG. 3A). The present inventors found that these myoepithelial cells represented a primitive level of myoepithelial differentiation because <1% of the cells expressed other myoepithelial markers such as Thy-1 (FIG. 3B, a). The staining for Thy-1 was performed as described in example 1 using the Thy-1 (AS0-2, Dianova, Hamburg, GmbH) as primary antibody, see also Table 4. However, if the cells were retained in a Thy-1 column, a myoepithelial-restricted subline could be generated which also expressed α-smooth muscle actin which is restricted to postmitotic myoepithelial cells in vivo (FIG. 3B). The staining for α-smooth muscle actin were performed as described in example 1. The present inventors reasoned that, if K18⁺ cells were also precursor cells of a lineage-restricted progeny within the luminal compartment, they could further mature within this compartment to differentiated cells. Sialomucin-expressing cells were eliminated by retention on a sialomucin-retaining column, but evidence for spontaneous maturation into sialomucin-positive cells was provided by the reccurrence of these cells within 2 weeks (FIG. 3C, a). These cells in turn could be retained in a similar column and kept as lineage-restricted in high-density cultures in the presence of serum (FIG. 3C, b, c). Taken together, these observations provide evidence for the existence of a suprabasal, multipotent epithelial cell belonging to the luminal epithelial lineage that can give rise to differentiated myoepithelial and luminal epithelial cells, and their precursors.

### Example 4

Only Suprabasal-Derived Epithelial Cells Give Rise to Terminal Duct Lobular Units (TDLU)

In the mouse and rat, the standard criteria for the presence of stem cells has been the ability to regenerate the entire structure of the mammary gland upon reimplantation of cells in syngeneic gland-free fat pads (Smith and Medina 1988). The present inventors performed a similar test in a three-dimensional laminin-rich gel as previously described (Petersen et al. 1992). Briefly, for three-dimensional cultures, 2.5×10⁵ luminal-derived, suprabasal-derived, and myoepithelial-derived cell lines were plated separately inside rBM (Matrigel®, lot# 40230A, Becton Dickinson, Mass.). Experiments were carried out in 24 well dishes (Nunc, Roskilde, Denmark) using 300 μl Matrigel in which single cells were suspended. Primary luminal epithelial cells, myoepithelial cells and uncultured terminal duct lobular unit (TDLU) organoids from the breast were used as control (Petersen et al. 1992). The percentage of TDLU formation defined by branching of cell clusters was quantified by phase contrast microscopy using a 10× objective and a 10× eye piece.

In this test the luminal-derived epithelial cell line or the myoepithelial-derived cells yielded morphologies very similar to what has already been described for primary breast cells (Petersen et al. 1992) (FIG. 4A). In contrast, embedding the suprabasal-derived epithelial cells gave rise to formation of larger, more elaborate morphologies resembling the entire functional unit of the breast gland, i.e. the terminal duct lobular unit (TDLU) (FIG. 4B and C). A similar frequency of TDLU structures (73%) was recorded if suprabasal cells were freshly prepared from primary cultures from two different biopsies. Sections of laminin-rich gels embedded with the suprabasal-derived epithelial cells and stained for differentiation markers of normal breast revealed a remarkable similarity to TDLU 's in vivo (FIG. 4D).

To provide evidence that this ability to generate a suprabasal-derived epithelial cell line from the human breast with all the above mentioned stem cell characteristics was not simply of this particular cell isolation the present inventors repeated the entire protocol including immortalisation of cells from a different biopsy twice, and the present inventors were able to reproduce the TDLU assay. Collectively, these data are in strong support of “suprabasal” cells within the luminal epithelial lineage as precursors of the TDLU, the functional units in the human breast.

### Example 5

The Suprabasal-Derived Cells are Keratin K19⁺ Similar to a Subpopulation of Cells in TDLU and Neoplastic Breast Epithelial Cells in vivo

To identify a candidate subpopulation within TDLU in which the stem cell could reside, the present inventors performed an analysis of the luminal epithelial markers expressed by the two established cell lines.

Cells were stained for keratin K19 as described in example 1 but using keratin K19 (BA17 and RCK108, DAKO, Glostrup, Denmark) as primary antibody, see also Table 4. Thus, keratin K19 was identified as a distinctive trait expressed only by the suprabasal-derived epithelial cells (Table 1). This difference in phenotype was confirmed further by reverse transcription PCR (RT-PCR), immunoblotting and immunostaining (FIG. 5A-C).

RNA isolation and reverse transcription PCR were performed as follows: Total RNA was extracted from monolayer cultures with Trizol® according to the manufacturer's instructions (Life Technologies, Tåstrup, Denmark). DNase-treated (DNase I Amp Grade, Life Technologies, Tåstrup, Denmark) total RNA (1.3 μg) was used as template for first strand synthesis with oligo dT primers (SuperScript First-Strand Synthesis System for RT-PCR, Life Technologies, Tåstrup, Denmark) in a 20 μl volume. A volume of 1 μl from this cDNA served as template for the subsequent PCR-amplifications in a PE 9700 thermal cycler with a heated lid (Applied Biosystems, Nœrum, Denmark), using primers (purchased from TAG Copenhagen, Copenhagen, Denmark) specific for HPV16 E6 and E7 (HPV16 E6 and HPV16 E7, respectively), keratin K19 (K19), sialomucin (MUC1), α-smooth muscle actin (αSM Actin) and glyceraldehyde 3-phosphate dehydrogenase (GAPDH). The primer sequences, annealing temperature (T_{A}) and number of amplification cycles for each reaction, as well as the resulting product size are listed below (Table 2). Each PCR-reaction was initiated with a 15 min incubation step at 95° C., followed by the specified number of cycles with denaturation at 94° C., annealing at the specified T_{A}, and extension at 72° C., for 1 min each, followed by a final extension step at 72° C. for 7 min. Each reaction was performed in a 50 μl volume containing 2.5U HotStar taq polymerase (Qiagen, KEBO Lab A/S, Albertslund, Denmark), 10×PCR buffer including MgCl₂ (Qiagen, KEBO Lab A/S, Albertslund, Denmark), 200 μM dNTP (Roche, Hvidovre, Denmark) and 200 nM of forward and reverse primers.

Control amplification was performed on RNA samples not subjected to reverse transcription to verify that no contaminating genomic DNA was present (data not shown). The PCR products were analysed by electrophoresis in 1.5% agarose gels (GibcoBRL, Life Technologies, Tåstrup, Denmark).

Brieflly, the immunoblotting was performed by lysing semi-confluent T-25 flasks of luminal- and suprabasal-derived epithelial cells, and T47D breast cancer cells (positive control;) for 30 min at 4° C. in buffer containing 1% Triton X-100 (Merck, Darmstadt, Germany), 1% deoxycholic acid (Sigma-Aldrich, Vallensbœk, Denmark), 10% glycerol (Apotek Austurbœjar, Reykjvik, Iceland), 20 mM Tris-HCl (USB, Cleveland, Ohio, US), pH 7.5,150 mM NaCl (Merck, Darmstadt, Germany), 2.5 mM EDTA (Titriplex II, Merck, Darmstadt, Germany), 1 mM PMSF(phenylmethylsulfonyl flouride, Sigma-Aldrich, Vallensbœk, Denmark), 1% aprotinin (trasylol, Sigma-Aldrich, Vallensbœk, Denmark), 100 μM NaVO₃ (Sigma-Aldrich, Vallensbœk, Denmark). The lysates were centrifuged and samples were subjected to 12% SDS-PAGE and run at 35 mV for 4 hours. The loading of lanes was equilibrated based on protein determinations by the Bio-Rad protein assay (Bio-Rad Laboratories, Hercules, Calif.). The samples were electrophoretically transferred to Immun-Blot PVDF Membrane (BioRad Laboratories, Hercules, Calif.) at 400 mA for 3-4 h at 4° C. in 20% methanol (Merck, Darmstadt, Germany), 0.2 M glycine (USB, Cleveland, Ohio, US), and 25 mM Tris-HCl (USB, Cleveland, Ohio, US). Blots were blocked in phosphate-buffered saline containing 5% dried milk (Osta og Smjörsalan, Reykjavik, Iceland) and 0.05% Tween-20 (USB, Cleveland, Ohio, US) for 1 h at room temperature before probing with anti-keratin K19. The blots were washed three times for 10 min in phosphate-buffered saline containing 0.05% Tween-20 (USB, Cleveland, Ohio, US) and then incubated with the anti-mouse IgG, horseradish peroxidase linked whole antibody (NA931, Amersham Pharmacia Biotech, Amersham, UK). After washing, bound antibodies were visualized using the ECL immunoblotting detection system (Amersham Pharmacia Biotech, Amersham, UK).

Immunostaining was preformed as described previously. Primary antibodies directed against keratin K19 (BA17 and RCK108, DAKO, Glostrup, Denmark) were used, see also Table 4.

In tissue sections of normal breast the present inventors found a limited expression of keratin K19 including staining of suprabasal cells (FIG. 6a, arrow), whereas in the majority of breast carcinomas, the neoplastic epithelial cells stain positive for keratin K19 (FIG. 6b). If the keratin K19⁺ cells were indeed potential stem cells, then the suprabasal-derived epithelial cell line should show evidence of multipotency with regard to keratin K19 expression. As seen in FIG. 7A, clones could be identified which diversified into both K14⁺ and K19⁺ cells. Similarly, cloning in laminin-rich gels also resulted in formation of TDLU structures, which showed correct segregation of cells into suprabasally/luminally positioned K19⁺ cells and basally located K14⁺ cells (FIG. 7B).

Finally, to provide yet further evidence for the morphogenic potential of these cells in vivo, the present inventors inoculated the fat pad of nude mice after preembedding the cells in a mixture of collagen gel and rBM (Yang et al. 1994). Briefly, luminal- and suprabasal-derived epithelial cells were inoculated subcutaneously into BALB/C nude mice after preembedding 106 cells in 500 μl of a mixture of collagen and rBM (20% Matrigel® (lot# 40230A, Becton Dickinson, Mass.)/80% collagen (Vitrogen-100, Cohesion, Palo Alto, Calif.)) (Yang et al. 1994). The mice were sacrificed after one week and the implants were sectioned and stained (see above).

The present inventors used this assay to show that the suprabasal-derived epithelial cell line segregated into suprabasal/luminal keratin K19⁺ cells and basal keratin K14⁺ cells (FIG. 7C).

It is concluded that the suprabasal-derived epithelial cells have an easily identifiable equivalent in vivo which appears to be identical to the stem cell of normal human breast.

The experiments described here establish both the existence of candidate multipotent stem cells in the human breast and the fact that they can be immortalised without loss of stem cell potential. The experiments also outline a method for their isolation and further characterisation. The multipotent cell line was derived from a suprabasally located cell in vivo which nevertheless belongs to the luminal epithelial lineage as evidenced by expression of ESA, claudin-1, keratins K18 and K19, but also by the ability to form monolayers that display a high TER. Embedding clonal populations in a three dimensional basement membrane gel or in mammary fat pads of mice reveal that the suprabasal-derived cell line recapitulates an elaborate morphology closely reminiscent of TDLU in vivo. The present inventors propose that cells with a suprabasal position within the luminal epithelial lineage (K19⁺, ESA⁺, MUC⁻) are candidate breast stem cells and putative precursors to human breast TDLUs.

TABLE 1
Keratin K19 is a distinctive trait expressed only by suprabasal-derived
epithelial cells as revealed by immunocytochemical staining.

| | Luminal-derived | Suprabasal-derived epithelial |
|---|---|---|
| Differentiated trait | epithelial cells | cells |
| Claudin-1 | + | + |
| Occludin | + | + |
| epithelial-specific | + | + |
| antigen (ESA) | | |
| Keratin K18 | + | + |
| Keratin K19 | ÷ | + |
| sialomucin (MUC) | + | ÷/+ |
| E-cadherin | + | + |

See table 4 for description of primary antibodies.

TABLE 2
Primer sequences

| | | | Amplification | Product Size |
|---|---|---|---|---|
| Primer | Sequence (5′-3′ direction) | T_{A} (C) | cycles | (bp) |
| HPV16 E6-FW | GCAACAGTTACTGCGACGTG | 55° | 30 | 234 |
| HPV16 E6-RV | GGACACAGTGGCTTTTGACA | | | |
| HPV16 E7-FW | GATGGTCCAGCTGGACAAGC | 55° | 30 | 143 |
| HPV16 E7-RV | GTGCCCATTAACAGGTCTTC | | | |
| K19-FW | GAGGTGGATTCCGCTCCGGGCA | 58° | 25 | 462 |
| K19-RV | ATCTTCCTGTCCCTCGAGCAG | | | |
| MUC1-FW | GTACCATCAATGTCCACGAC | 60° | 30 | 351 |
| MUC1-RV | CTACGATCGGTACTGCTAGG | | | |
| αSM Actin-FW | GGAATCCTGTGAAGCAGCTC | 56° | 32 | 1200 |
| αSM Actin-RV | CACAGTTGTGTGCTAGAGACAGAG | | | |
| GAPDH-FW | GAAGGTGAAGGTCGGAGT | 54° | 25 | 226 |
| GAPDH-RV | GAAGATGGTGATGGGATTTC | | | |

Primers were specific for human papilloma virus 16 E6 and E7 (HPV16 E6 and HPV16 E7, respectively), keratin K19 (K19), Sialomucin (MUC1), α smooth muscle actin (αSM Actin) and glyceraldehyde 3-phosphate dehydrogenase (GAPDH). FW: forward primer; RV: reverse primer.

TABLE 3
Biopsy P595 (D492, suprabasal cell line)

| Exp no. | Passage | MACS | Media | Immortalized |
|---|---|---|---|---|
| D470 | 1 | | CDM3 | |
| D480 | 2 | MAM6− | § + CT + EGF + | |
| | | | KGF | |
| D486 | 3 | β4+ | § + 10% FCS + | |
| | | | I + H + EGF | |
| D492 | 3 | | § + 10% FCS + | X |
| | | | I + H + EGF | |
| | 6 | | H14 | |
| | 27 | ESA+ | H14 | |

Biopsy P594 (D490, suprabasal cell line)

| Exp. no | Passage | MACS | Media | Immortalized |
|---|---|---|---|---|
| D467/1 | 1 | | CDM3 | |
| D467/8 | 1 | | § + 10% FCS + I + | |
| | | | H + EGF | |
| D467/6-7 | 1 | | § + CT + EGF | |
| 475/2 | 2 | MAM6− | § + 10% FCS + | |
| | | | I + H + EGF | |
| 477/1 | 2 | β4+ | § + 10% FCS + | |
| | | | I + H + EGF | |
| 478/1 | 2 | β4+ | § + CT + EGF | |
| D490/1 | 3 | MAM6− | § + 10% FCS + | |
| | | | I + H + EGF | |
| D500(D490/1) | 3 | | § + 10% FCS + | X |
| | | | I + H + EGF | |
| | 6 | | H14 | |
| | 10 | ESA+ | H14 | |

Biopsy A245 (TH69, suprabasal cell line)

| Exp. no | Passage | MACS | Media | Immortalized |
|---|---|---|---|---|
| A245 | 1 | | CDM3 | |
| TH65 | 2 | MAM6− | CDM3 | |
| TH68 | 3 | ESA+ | CDM3 | |
| TH69 | 3 | | CDM3 | X |
| | 3 | | CDM3 + 10% FCS | |
| | 4 | | CDM3 | |
| | 6 | | H14 | |

Biopsy P591 (D382, luminal cell line)

| Exp no. | Passage | MACS | Media | Immortalized |
|---|---|---|---|---|
| D346 | 1 | | CDM3 | |
| D360 | 2 | MAM6+ | CDM6 | |
| D369 | 3 | MAM6+ | CDM6 | |
| D382 | 3 | | CDM3 | X |
| | 11 | | H14 | |

Biopsy A253 (TH82, primary suprabasal cells)

| Exp no. | Passage | MACS | Media |
|---|---|---|---|
| A253 | 1 | | CDM3 |
| TH78 | 2 | MAM6− | CDM3 |
| TH82* | 3 | ESA+ | |

Biopsy A269 (TH95, primary suprabasal cells)

| Exp no. | Passage | MACS | Media |
|---|---|---|---|
| A269 | 1 | | CDM3 |
| TH90 | 2 | MAM6− | CDM3 |
| TH95* | 3 | ESA+ | |

ABREVIATIONS USED IN TABLE 3:
Exp no.: number of experiment.
MACS: MiniMACS magnetic cell separation system.
MAM6−: flow-through of an anti-sialomucin column.
MAM6+: retention in an anti-sialomucin column.
β4+: retention in a β4-integrin column.
ESA+: retention in an anti-ESA column.
§: DMEM/F-12 medium 1:1 supplemented with glutamine, 2 mM final.
CDM3: CDM3 medium (Petersen and van Deurs 1987)
CDM6: CDM6 medium (Pechoux et al. 1999)
H14: H14 medium (Blaschke et al. 1994)
CT: cholera toxin (10 ng/ml final).
EGF: epidermal growth factor (100 ng/ml final).
KGF: keratinocyte growth factor (10 μg/ml final).
I: insulin (3 μg/ml final).
H: hydrocortisone (1.4 × 10⁶ M final).
FCS: fetal calf serum (E.C. approved, virus and mycoplasma tested).
*After ESA purification cells were immediately embedded into 300 μl Matrigel ® and cultured for 2 weeks in CDM3 medium.

TABLE 4
Primary antibodies.

| antibody directed against | antibody description (name and provider) |
|---|---|
| keratin K18 | F3006; Trichem Aps, Denmark, |
| keratin K19 | BA17, DAKO, Glostrup, Denmark, |
| keratin K19 | RCK108, DAKO, Glostrup, Denmark, |
| sialomucin | MAM6, clone 115D8, Biogenesis Ltd., |
| | Poole, UK, |
| occludin | OC-3F10, Zymed Laboratories, San |
| | Francisco, CA, |
| polyclonal claudin-1 | Zymed Laboratories, San Francisco, CA, |
| epithelial-specific antigen | ESA; VU-1D9, NovoCastra, Newcastle |
| | upon Tyne, UK, |
| E-cadherin | HECD-1, kindly provided by Dr. Atsushi |
| | Ochiai, Tokio, Japan, |
| Thy-1 | AS0-2, Dianova, Hamburg, GmbH, |
| α-smooth muscle actin | 1A4, Sigma-Aldrich, Vallensbæk, Denmark, |
| vimentin | V9, DAKO, Glostrup, Denmark, |
| α1 chain of laminin-1 | EB7, kindly provided by I. Virtanen, |
| | University of Helsinki, |
| keratin K14 | LL002, NovoCastra, Newcastle upon Tyne, |
| | UK. |

### References

Anbazhagan, R., Osin, P. P., Bartkova, J., Nathan, B., Lane, E. B., and Gusterson, B .A. 1998. The development of epithelial phenotypes in the human fetal and infant breast. J. Pathol. 184: 197-206.
Ausubel et al. Current protocols in molecular biology, (2000) John Wiley and Sons, inc., N.Y.
Balmain A, Harris C C. 2000. Carcinogenesis in mouse and human cells: parallels and paradoxes. Carcinogenesis. 21:371-7.
Band, V. 1995. Preneoplastic transformation of human mammary epithelial cells. Semin. Cancer Biol. 6: 185-92.
Blaschke, R. J., A. R. Howlett, P.-Y. Deprez, O. W. Petersen, and M. J. Bissell. 1994. Cell differentiation by extracellular matrix components. Meth. Enzymol. 245: 535-556.
Bouwens, L. 1998. Cytokeratins and cell differentiation in the pancreas. J. Pathol. 184: 234-9.
Fridmacher, V., Le Bert, M., Guillou, F., and Magre, S. 1995. Switch in the expression of the K19/K18 keratin genes as a very early evidence of testicular differentiation in the rat. Mech. Dev. 52: 199-207.
Gudjonsson, T., Villadsen, R., Nielsen, H. L., Rønnov-Jessen, L., Bissell, M. J., Petersen, O. W. 2002a. Isolation, immortalization, and characterization of a human breast epithelial cell line with stem cell properties. Genes Dev. 16:693-706.
Gudjonsson, T., Rønnov-Jessen, L., Villadsen, R., Bissell, M. J., and Petersen, O. W. 2002b. Normal and tumor-derived myoepithelial cells differ in their ability to interact with luminal breast epithelial cells for polarity and basement membrane deposition. J. Cell Sci. 115:39-50.
Hudson, D. L., Guy, A. T., Fry, P., O'Hare, M. J., Watt, F. M., and Masters, J. R. 2001. Epithelial cell differentiation pathways in the human prostate: identification of intermediate phenotypes by keratin expression. J. Histochem. Cytochem. 49: 271-8.
Hudson, D. L., M. O'Hare, F. M. Watt, and J. R. Masters. 2000. Proliferative heterogeneity in the human prostate: evidence for epithelial stem cells. Lab Invest 80: 1243-50.
Katakura et al., 1998, Methods Cell Biol., 57, 69-91
Michel, M., Torok, N., Godbout, M. J., Lussier, M., Gaudreau, P., Royal, A., and Germain, L. 1996. Keratin 19 as a biochemical marker of skin stem cells in vivo and in vitro: keratin 19 expressing cells are differentially localized in function of anatomic sites, and their number varies with donor age and culture stage. J. Cell Sci. 109: 1017-28.
Péchoux, C., Gudjonsson, T., Rønnov-Jessen, L., Bissell, M. J., and Petersen, O. W. 1999. Human mammary luminal epithelial cells contain progenitors to myoepithelial cells. Dev. Biol. 206: 88-99.
Petersen, O. W. and B. van Deurs. 1987. Preservation of defined phenotypic traits in short-time cultured human breast carcinoma derived epithelial cells. Cancer Res. 47: 856-866.
Petersen, O. W., L. Rønnov-Jessen, A. R. Howlett, and M. J. Bissell. 1992. Interaction with basement membrane serves to rapidly distinguish growth and differentiation pattern of normal and malignant human breast epithelial cells. Proc. Natl. Acad. Sci. USA 89: 9064-9068.
Petersen, O. W., Rønnov-Jessen, L., Weaver, V. M., and Bissell, M. J. 1998. Differentiation and cancer in the mammary gland: Shedding new light on an old dichotomy. Adv. Cancer Res. 75: 135-161.
Rønnov-Jessen, L., J. E. Celis, B. van Deurs, and O. W. Petersen. 1992. A fibroblast-associated antigen: Characterization in fibroblasts and immunoreactivity in smooth muscle differentiated stromal cells. J. Histochem. Cytochem. 40: 475-486.
Rønnov-Jessen, L., O. W. Petersen, V. E. Koteliansky, and M. J. Bissell. 1995. The origin of the myofibroblasts in breast cancer: Recapitulation of tumor environment in culture unravels diversity and implicates converted fibroblasts and recruited smooth muscle cells. J. Clin. Invest. 95: 859-873.
Rønnov-Jessen, L., Petersen, O. W., and Bissell, M. J. 1996. Cellular changes involved in conversion of normal to malignant breast: The importance of the stromal reaction. Physiol. Rev. 76: 69-125.
Santner, S. J., Dawson, P. J., Tait, L., Soule, H. D., Eliason, J., Mohamed, A. N., Wolman, S. R., Heppner, G. H., and Miller, F. R. 2001. Malignant MCF10CA1 cell lines derived from premalignant human breast epithelial MCF10AT cells. Breast Cancer Res. Treat. 65: 101-10.
Sambrook et al., Molecular Cloning, A Laboratory Manual, 2nd Edition (1989) Cold Spring Harbor Laboratory Press, Cold Spring Harbor, N.Y.
Smalley, M. J., Titley, J., Paterson, H., Perusinghe, N., Clarke, C., and O'Hare, M. J. 1999. Differentiation of separated mouse mammary luminal epithelial and myoepithelial cells cultured on EHS matrix analyzed by indirect immunofluorescence of cytoskeletal antigens. J. Histochem. Cytochem. 47: 1513-24.
Smith, G. H. 1996. Experimental mammary epithelial morphogenesis in an in vivo model: Evidence for distinct cellular progenitors of the ductal and lobular phenotype. Breast Cancer Res. Treat. 39: 21-31.
Smith, G. H. and Chepko, G. 2001. Mammary epithelial stem cells. Microscopy Res. Techn. 52: 190-203.
Smith, G. H. and Medina, D. 1988. A morphologically distinct candidate for an epithelial stem cell in mouse mammary gland. J. Cell Sci. 89: 173-183.
Spancake, K. M., Anderson, C. B., Weaver, V. M., Matsunami, N., Bissell, M. J., and White, R. L. 1999. E7-transduced human breast epithelial cells show partial differentiation in three-dimensional culture. Cancer Res. 59: 6042-5.
Stampfer, M. R., Garbe, J., Levine, G., Lichtsteiner, S., Vasserot, A. P., and Yaswen, P. 2001. Expression of the telomerase catalytic subunit, hTERT, induces resistance to transforming growth factor beta growth inhibition in p16INK4A(−) human mammary epithelial cells. Proc. Natl. Acad. Sci. USA 98: 4498-503.
Stingl, J., Eaves, C. J., Kuusk, U., and Emerman, J. T. 1998. Phenotypic and functional characterization in vitro of a multipotent epithelial cell present in the normal adult human breast. Differentiation 63: 201-213.
Stingl, J., Eaves, C. J., Zandieh, I., and Emerman, J. T. 2001. Characterization of bipotent mammary epithelial progenitor cells in normal adult human breast tissue. Breast Cancer Res. Treat. 67: 93-109.
Stosiek, P., Kasper, M., and Karsten, U. 1990. Expression of cytokeratin 19 during human liver organogenesis. Liver 10: 59-63.
Wazer, D. E., Liu, X.-L., Chu, C., Gao, Q., and Band, V. 1995. Immortalization of distinct human mammary epithelial cell types by human papilloma virus 16 E6 or E7. Proc. Natl. Acad. Sci. USA 92. 3687-3691
Willey, J. C., Broussoud, A., Sleemi, A., Bennett, W. P., Cerutti, P., and Harris, C. C. 1991. Immortalization of normal human bronchial epithelial cells by human papillomaviruses 16 or 18. Cancer Res. 51: 5370-7.
Yang, I., Guzman, R. C., Popnikolov, N., Bandyopadhyay, G. K., Christov, K., Collins, G., and Nandi, S. 1994. Phenotypic characterization of collagen gel embedded primary human breast epithelial cells in athymic nude mice. Cancer Lett. 81: 117-127.
Zwerschke, W. and Jansen-Durr, P. 2000. Cell transformation by the E7 oncoprotein of human papillomavirus type 16: interactions with nuclear and cytoplasmic target proteins. Adv. Cancer Res. 78: 1-29.

1. An isolated cell derived from luminal epithelial cells of a mammary gland which is capable of proliferating and differentiating into cells of mammary gland luminal epithelial and myoepithelial cell lineages said isolated cell being capable of forming a cell culture comprising cells which are positive staining for the luminal epithelial marker ESA (ESA+) and negative staining for sialomucin (MUC−), so-called (ESA+/MUC−) cells.

2. A cell according to claim 1, which is isolated from suprabasal luminal epithelial cells of the mammary gland.

3. A cell according to claim 2, which is a human cell.

4. A cell according to claim 1, which is immortalised.

5. A cell population composed of cells according to claim 1.

6. An immortalised cell line derived from the cell of claim 4.

7. An immortalised cell line according to claim 6, wherein the immortalising step comprises transfecting the cells with a nucleic acid molecule encoding an immortalising polypeptide.

8. An immortalised cell line according to claim 7, wherein the immortalising step comprises transfecting the cells with a nucleic acid molecule encoding a papillomavirus polypeptide selected from the group consisting of E6, E7 and a nucleic acid molecule comprising E6 and E7.

9. An immortalised cell line according to claim 7, wherein the immortalising step comprises transforming the cells with at least one retroviral vector including an expression cassette comprising a nucleic acid molecule encoding a papillomavirus polypeptide selected from the group consisting of E6, E7 and a nucleic acid molecule comprising E6 and E7, and selecting the immortalised cells.

10. An immortalised cell line according to claim 9, wherein the immortalising step is performed by transforming the cells with retrovirus-containing supernatant from the PA317 LXSN HPV16E6E7 cell line and selecting the immortalised cells.

11. An immortalised cell line according to claim 6 that in culture is capable of forming branching structures resembling terminal duct lobular units of the mammary gland in morphology and/or by marker expression.

12. An immortalised cell line according to claim 6 which comprises cells that are positive staining for the keratin K19.

13. An immortalised cell line according to claim 6 that is derived from a cell selected from the group consisting of a rodent cell, a porcine cell, a ruminant cell, a bovine cell, a caprine cell, a equine cell, a canine cell, a ovine cell, a feline cell and a primate cell.

14. An immortalised cell line according to claim 13 that is selected from the group consisting of cells from mice, rats and rabbits.

15. An immortalised cell line according to claims 13 that is a human cell line.

16. The immortalised cell line according to claim 6 which is deposited in accordance with the provisions of the Budapest Treaty on the International Recognition of the Deposit of Microorganisms for the Purposes of Patent Procedure at Deutsche Sammlung von Mikroorganismen und Zellkulturen GmbH (DSMZ) and has obtained the accession number DSM ACC 2529.

17. A method for isolation of an at least bi-potent mammary gland tissue cell, comprising the steps of:
(i) separating said tissue into two or more different cell types
(ii) culturing each of said different cell types under cell differentiation conditions and
(iii) selecting the cell type(s) that is/are capable of differentiating into at least two morphologically and/or phenotypically different cell types.

18. A method according to claim 17 in which the at least bi-potent cell is a cell according to an isolated cell derived from luminal epithelial cells of a mammary gland which is capable of proliferating and differentiating into cells of mammary gland luminal epithelial and myoepithelial cell lineages said isolated cell being capable of forming a cell culture comprising cells which are positive staining for the luminal epithelial marker ESA (ESA+) and negative staining for sialomucin (MUC−), so-called (ESA+/MUC−) cells.

19. A method for testing the toxic effect, if any, of a substance on mammary gland epithelial cells, the method comprising:
(i) culturing or maintaining the cells of claim 1 in a non-toxic medium;
(ii) adding the substance to be tested to the medium; and
(iii) determining the response, if any, of the cells, including changes in cell growth rate, cell death rate, apoptosis, cell metabolism, inter- as well as intra-cellular communication, morphology, mRNA or protein expression and antigen expression.

20. A method for testing the carcinogenic effect, if any, of a substance on mammary gland epithelial cells, the method comprising:
(i) culturing the cells of claim 1 in a growth medium which maintains the cells as non-transformed cells;
(ii) adding the agent, compound or factor under test to the cell culture; and
(iii) determining the neoplastic response, if any, of the so contacted cells by changes in morphology, tumorigenicity in animals, mRNA expression and/or antigen expression as well as other changes which is associated with carcinogenicy.

21. A method as claimed in claim 20, wherein the tumorigenicity test comprise the introduction of said treated cells into an immune incompetent test animal.

22. A method of testing the ability, if any, of a substance to modulate the differentiation of non-terminal differentiated mammary gland epithelial cells, the method comprising:
(i) culturing or maintaining the cells of claim 1 in a medium which in tiself does not modulate the differentiation;
(ii) adding the substance under test to the cell culture; and
(iii) determining the differentiation modulation responses, if any, of the so contacted cells by changes in cell growth rate, cell death rate, apoptosis, cell metabolism, inter- as well as intra-cellular communication, morphology, mRNA or protein expression or antigen expression as well as other changes which is associated with differentiation.

23. A method for screening a substance for its ability, if any, to interact with a cellular protein, the method comprising:
(i) transfecting a cell of claim 1 with a gene construct enabling transfected cells to express said protein;
(ii) adding the substance to be tested to the cells; and
(iii) determining the interaction, if any, with a cellular protein by changes in cell growth rate, cell death rate, apoptosis, cell metabolism, inter- as well as intra-cellular communication, morphology, mRNA or protein expression, antigen expression or other changes which either directly or indirectly is supposed to be associated with said protein.

24. A method according to claim 23 in which said cellular protein is selected from the group consisting of estrogen receptor-alpha, estrogen receptor-beta and progesterone receptor.

25. A method of transplanting a vertebrate host with a cell according to claim 1, comprising the step of introducing the cell into the vertebrate host.

26. A method of in vivo administration of a protein or gene of interest to an individual in need thereof, comprising the step of transfecting the cell-population of claim 1 with a vector comprising DNA or RNA which expresses the protein or gene of interest and introducing the transfected cell into said individual.

27. Use a cell according to claim 1 to prevent and/or treat cellular debilitations, derangements and/or dysfunctions and/or other disease states in mammals, comprising administering to a mammal a therapeutically effective amount of said cells, or cells or tissues derived therefrom.

28. A method of tissue repair or transplantation in mammals, comprising administering to a mammal a therapeutically effective amount of a cell according to claim 1, or cells or tissues derived therefrom.

29. A pharmaceutical composition comprising: a therapeutically effective amount of a cell according to claim 1, or cells or tissues derived therefrom; and a pharmaceutically acceptable carrier.

30. The pharmaceutical composition of claim 29 further comprising a proliferation factor or lineage commitment factor.

31. A diagnostic agent comprising the cell of claim 1, or any part thereof
